(19) United States
(12) Patent Application Publication
(10) Pub. No.: US 2024/0294587 A1
(43) Pub. Date: Sep. 5, 2024

(54) K-RAS INHIBITOR

(71) Applicant: THE JOHNS HOPKINS UNIVERSITY, Baltimore, MD (US)

(72) Inventors: Miho Iijima, Balitmore, MD (US); Hiroshi Senoo, Baltimore, MD (US); Hiromi Sesaki, Baltimore, MD (US)

(21) Appl. No.: 18/551,557

(22) PCT Filed: Mar. 21, 2022
(86) PCT No.: PCT/US22/21077

§ 371 Date: Sep. 20, 2023

Related U.S. Application Data
(60) Provisional application No. 63/164,127, filed on Mar. 22, 2021.

(51) Int. Cl. C07K 14/47; A61K 38/00; A61P 3/08

(52) U.S. Cl. CPC C07K 14/4702; A61P 3/08; A61K 38/00

(57) ABSTRACT

The present disclosure provides peptide inhibitors of K-Ras, and polynucleotides, compositions, and methods of use thereof.

### CROSS-REFERENCE TO RELATED APPLICATIONS

This application claims the benefit of U.S. Provisional Application Nos. 63/164,127, filed Mar. 22, 2021, the content of which is herein incorporated by reference in its entirety.

### STATEMENT REGARDING FEDERALLY-SPONSORED RESEARCH OR DEVELOPMENT

This invention was made with government support under grants GM131768 and GM084015 awarded by the National Institutes of Health. The government has certain rights in the invention.

### FIELD

The present disclosure provides peptide inhibitors of K-Ras and methods of use thereof.

### SEQUENCE LISTING STATEMENT

The text of the computer readable sequence listing filed herewith, titled “39305-601-SEQUENCE_LISTING_ST25”, created Mar. 21, 2022, having a file size of 2,187 bytes, is hereby incorporated by reference in its entirety.

### BACKGROUND

Ras proteins (K-Ras, H-Ras, and N-Ras) are ubiquitously expressed in almost all types of cells and function as signal transducers between cell membrane-based growth factor signaling and the MAPK pathways. The Ras proteins play a critical role in the regulation of cell proliferation, differentiation, and survival. Dysregulation of Ras signaling pathways is almost invariably associated with disease. Hyper-activating somatic mutations in Ras are among the most common found in human cancer. Although mutation of any one of the three Ras proteins has been shown to lead to oncogenic transformation, K-Ras mutations are by far the most common in human cancer. For example, K-Ras mutations are known to be often associated with pancreatic, colorectal, and non-small-cell lung carcinomas.

### SUMMARY

Disclosed herein are peptides, or pharmaceutically acceptable salts or solvates thereof, comprising an amino acid sequence with at least 50% similarity to SEQ ID NO: 1. Also disclosed are polynucleotides comprising a nucleic acid sequence encoding the peptides disclosed herein and composition comprising the peptides polynucleotides disclosed herein.

Further disclosed are methods of treating a disease or disorder comprising administering to a subject in need thereof an effective amount of a peptide, polynucleotide or composition disclosed herein. In some embodiments, the disease or disorder is characterized by hyperactive K-Ras or abnormal K-Ras signaling. In some embodiments, the disease or disorder comprises cancer or a developmental disorder.

Further disclosed are methods for modulating the activity of a Ras protein comprising contacting the Ras protein with an effective amount of a peptide, polynucleotide or composition disclosed herein.

Other aspects and embodiments of the disclosure will be apparent in light of the following detailed description and accompanying figures.

### BRIEF DESCRIPTION OF THE DRAWINGS

FIGS. 1A-1H show that insulin induces the formation of KARATE. HEK293 cells expressing FLAG-mTOR were subjected to serum starvation for 5 h and then incubated with or without 100 nM insulin for 30 min. Cells were lysed and subjected to immunoprecipitation using anti-FLAG beads. FIG. 1A is a Western blot of the analysis of the cell lysates and pellet fractions. HEK293 cells carrying FLAG-RAPTOR or FLAG-RICTOR were subjected to immunoprecipitation using anti-FLAG beads with or without insulin stimulation after serum starvation. FIG. 1B is a Western blot of the analysis of the cell lysates and pellet fractions. Parental WT and RhoA-KO HEK293 cells carrying FLAG-RICTOR were subjected to immunoprecipitation with or without insulin stimulation after serum starvation. FIG. 1C is a Western blot of the analysis of the cell lysates and pellet fractions. Parental WT and RhoA-KO HEK293 cells were lysed and subjected to immunoprecipitation using anti-RhoA antibodies. FIG. 1D is a Western blot of the analysis of the cell lysates and pellet fractions. Parental WT and K-Ras-KO HEK293 cells were subjected to immunoprecipitation using anti-RhoA antibodies. FIG. 1E is a Western blot of the analysis of the cell lysates and pellet fractions. GFP-RhoA, FLAG-K-Ras4A, and FLAG-K-Ras 4B were purified from a Dictyostelium bioreactor. These proteins were loaded with GDP or GTPγS in vitro. GFP-RhoA was then incubated with FLAG-K-Ras proteins and pulled down by GFP-Trap beads. FIG. 1F is a Western blot of pellets analyzed with antibodies to FLAG and GFP. GFP-RhoA was incubated with a constitutively GTP-bound K-Ras 4B_{G12V} and pulled down by GFP-Trap beads. FIG. 1G is a Western blot of pellets. FIG. 1H is a schematic of complexation of GTP-K-Ras 4B, GDP-RhoA, and mTORC2 in response to insulin. All of the experiments were repeated at least three times.

FIGS. 2A-2D show purified KARATE phosphorylates AKT in vitro. FLAG-RAPTOR or FLAG-RICTOR was immunoprecipitated from HEK293 cells with or without insulin stimulation after serum starvation using anti-FLAG beads (FIG. 2A). The immunoprecipitants were incubated with inactive AKT and ATP. The phosphorylation of AKT at S473 was analyzed by Western blotting as shown in FIG. 1A. mTORC2 was immunopurified using FLAG-RICTOR from HEK293 cells without insulin stimulation. Purified mTORC2 was incubated with AKT and ATP in the presence or absence of GDP-loaded FLAG-RhoA and/or GTPγS-loaded FLAG-K-Ras 4B. AKT phosphorylation at S473 was analyzed by Western blotting (FIG. 2B). Immunopurified mTORC2 from HEK293 cells without insulin stimulation was incubated with AKT and ATP together with the indicated GFP-RhoA proteins and GTP-bound FLAG-K-Ras 4B_{G12V}. S473 AKT phosphorylation was analyzed by Western blotting (FIG. 2C). FIG. 2D is a schematic of a model of AKT phosphorylation by KARATE. All of the experiments were repeated at least three times.

FIGS. 3A-3H show that RhoA and K-Ras 4B interaction facilitates assembly and function of KARATE. FIG. 3A is the amino acid sequence of the C-terminal region of K-Ras 4B—the helix α5 is highlighted and K-tetracosapeptide is underlined. Its predicted structure is shown on the right: positively- and negatively-charged surfaces are highlighted. FLAG-tagged GTP-bound K-Ras 4B_{G12V} was incubated with GDP-loaded GFP-RhoA in the presence of K-tetracosapeptide (K-TC) or a scrambled peptide (50, 250, and 1250 nM). GFP-RhoA was pulled down using GFP-Trap beads. The pellets were analyzed by Western blotting using antibodies to FLAG and GFP (FIG. 3B) and quantified by band intensity (FIG. 3C). Bars are mean±SD (n=3). FIG. 3D is a schematic of the interaction of K-Ras 4B and RhoA in the presence of K-tetracosapeptide (K-TC) or a scrambled peptide. Immunopurified mTORC2 using FLAG-RICTOR from HEK293 cells without insulin stimulation was mixed with GTP-bound K-Ras 4B_{G12V} and GDP-loaded RhoA. The mixtures were incubated with inactive AKT and ATP in the presence of K-tetracosapeptide or scramble peptide (50, 250, and 1250 nM). S473 AKT phosphorylation was analyzed by Western blotting (FIG. 3E) and band intensity of phospho-AKT relative to total AKT was quantified (FIG. 3F). Bars are mean±SD (n=3). After serum starvation, HEK293 cells were stimulated by insulin. TAT-K-tetracosapeptide or a TAT peptide was included during scrum starvation and insulin stimulation. S473 AKT phosphorylation was analyzed by Western blotting. (FIG. 3G) and band intensity was quantified (FIG. 3H). Bars are mean±SD (n=3). One-way ANOVA with post-hoc Tukey was performed in (C, F and H): **p<0.01, ***p<0.001.

FIGS. 4A-4H show that PKA phosphorylates RhoA and K-Ras 4B and promotes their interaction. FLAG-K-Ras 4B or FLAG-K-Ras 4B_{S181A} were immunoprecipitated with anti-FLAG beads from HEK293 cells with or without insulin stimulation after serum starvation. S181 K-Ras 4B phosphorylation was examined by Western blotting (FIG. 4A). YFP-RhoA or YFP-RhoA_{S188A} was immunoprecipitated with GFP-Trap from HEK293 cells with or without insulin stimulation after serum starvation. S188 RhoA phosphorylation was analyzed in Western blotting (FIG. 4B). Purified GDP-loaded GFP-RhoA or GDP-loaded, phospho-defective GFP-RhoA_{S188A} was incubated with GTP-bound FLAG-K-Ras 4B_{G12V} or GTP-bound, phospho-defective FLAG-K-Ras 4B_{G12V,S181A} and pulled down by GFP-Trap. The pellets were analyzed by Western blotting (FIG. 4C). Immunoprecipitated mTORC2 by FLAG-RICTOR was incubated with the indicated GDP-loaded or GDP-loaded, phosphor-defective forms of FLAG-RhoA and GTP-bound or GTP-bound, phosphor-defective FLAG-K-Ras 4B and tested for AKT phosphorylation. Western Blots are shown in FIG. 4D. HEK293 cells expressing FLAG-K-Ras 4B or YFP-RhoA were serum-starved for 5 h. In the last 30 min, the indicated kinase inhibitors were individually added to the starvation medium (see Methods). Cells were then stimulated with 100 nM insulin for 30 min. FLAG-K-Ras 4B or YFP-RhoA were immunoprecipitated and analyzed for phosphorylation by Western blotting using antibodies to K-Ras and phospho-serine for K-Ras 4B and RhoA and phospho-RhoA (S188) for RhoA (FIG. 4E). Purified FLAG-K-Ras 4B or -K-Ras 4B_{S181A} (FIG. 4F) and Purified FLAG-RhoA or -RhoA_{S188A} (FIG. 4G) were incubated with the catalytic subunit of PKA in the presence or absence of ATP. Phosphorylation was analyzed by Western blotting. FIG. 4H is a schematic of a proposed model of PKA phosphorylation of RhoA and K-Ras 4B. All of the experiments were repeated at least three times.

FIGS. 5A-5I show that S188-phosphorylated, GDP-bound RhoA regulates AKT phosphorylation in cells. Parental WT and two independent RhoA-KO HEK293 cells were stimulated with insulin after serum starvation. The phosphorylation of AKT (S473) and its direct substrates were analyzed using Western blotting (FIG. 5A) and band intensity was quantified (FIG. 5B). Bars are mean±SD (n=3). FIG. 5C is a schematic of a proposed model for S188-phosphorylated, GDP-bound RhoA regulation of AKT phosphorylation. Parental WT and two independent K-Ras-KO HEK293 cells were treated with insulin after serum starvation. The phosphorylation of AKT (S473) was examined using Western blotting (FIG. 5D) and band intensity was quantified (FIG. 5E). Bars are mean±SD (n=3). WT, GDP-bound (T19N), or GTP-bound (Q63L) YFP-RhoA was expressed in RhoA-KO cells. Phosphorylation of AKT and MLC was examined by Western blotting (FIG. 5F) and band intensity was quantified (FIG. 5G). Bars are mean±SD (n=3). WT, phospho-defective (S188A), or phospho-mimetic (S188E) YFP-RhoA was expressed in RhoA-KO cells and tested for insulin-stimulated AKT phosphorylation by Western blotting (FIG. 5H) and quantification of band intensity (FIG. 5I). Bars are mean±SD (n=3). One-way ANOVA with post-hoc Tukey was performed in (B, E, G, and I): *p<0.05, **p<0.01. ***p<0.001. All of the experiments were repeated at least three times.

FIGS. 6A-6G show that KARATE controls insulin signaling in 3T3-L1 adipocytes. Differentiation into adipocytes was induced in WT 3T3-L1 cells and RhoA-KO 3T3-L1 cells expressing the indicated FLAG-RhoA proteins. Differentiation was analyzed Western blotting using anti-adiponectin antibodies (FIG. 6A) and band intensity was quantified (FIG. 6B). Bars are mean f SD (n=3). FIG. 6C is images of WT and RhoA-KO 3T3-L1 cells stained with oil red O before and after induction of the differentiation. FIG. 6D is images RhoA-KO 3T3-L1 cells expressing the indicated RhoA proteins and analyzed by oil red O stain after induction of differentiation. Differentiated WT 3T3-L1 adipocytes were stimulated with insulin after serum starvation in the presence of TAT-K-tetracosapeptide or a TAT peptide. Phosphorylation of AKT and TBC1D4 was analyzed by Western blotting (FIG. 6E) and quantification of band intensity (FIG. 6F). Bars are mean±SD (n=3). FIG. 6G is confocal microscopy images of differentiated WT 3T3-L adipocytes carrying GLUT4-GFP following stimulation by insulin for 30 min after serum starvation in the presence of TAT-K-tetracosapeptide or a TAT peptide. One-way ANOVA with post-hoc Tukey was performed in (B and F): *p<0.05, **p<0.01, ***p<0.001. All of the experiments were repeated at least three times.

FIGS. 7A and 7B are images of SDS-PAGE gels of purified human protein from a bioreactor using Dictyostelium cells: GFP-RhoA, FLAG-K-Ras4A, and FLAG-K-Ras 4B in FIG. 7A and FLAG-RhoA proteins and FLAG-K-Ras 4B proteins in FIG. 7B.

FIG. 8 is a Western blot showing that K-Ras 4B does not homo-dimerize upon insulin stimulation. HEK293 cells expressing YFP-RhoA or YFP-K-Ras 4B were subjected to immunoprecipitation using GFP-Trap beads with or without insulin stimulation after serum starvation. Cell lysates and pellet fractions were analyzed by Western blotting using antibodies to GFP. K-Ras and GAPDH. The experiments were repeated at least three times.

FIG. 9 is Western blots showing phosphorylation of human K-Ras 4B and RhoA in Dictyostelium cells. The indicated FLAG-K-Ras 4B and FLAG-RhoA proteins were expressed and purified from Dictyostelium cells with or without chemoattractant cAMP stimulation. FLAGK-Ras 4B and FLAG-RhoA that were purified after stimulation became phosphorylated at S181 and S188, respectively. The S181A mutation blocked the phosphorylation in K-Ras 4B, while the S188A mutation inhibited the phosphorylation in RhoA. The experiments were repeated at least three times.

FIG. 10 is a schematic of the phylogenetic tree of human Ras GTPases against the Dictyostelium RacC. The phylogenetic tree was created using Fast Minimum Evolution method at COBALT.

FIGS. 11A-11F show that human K-Ras 4B functionally replaces Dictyostelium RasC. WT cells expressing a GTP-bound form of FLAG-tagged human Ras proteins were stimulated with the chemoattractant cAMP (1 μM). Phosphorylation of PkbR1, PkbA, and Erk2 was analyzed by Western blotting (FIG. 11A) and band intensities were quantified (FIG. 11B). WT cells at 30 s were set at 100%. Values are average±SD (n=3). Expression levels of human Ras proteins were analyzed by Western blotting with antibodies to FLAG and actin (FIG. 11C). Phosphomimetic GDP-bound GFPRacE_{T25N,S192D} was incubated with GTP-bound FLAG-tagged human Ras proteins and pulled down using GFP-Trap (FIG. 11D). (E) FLAG-RacE_{T25N,S192D} and FLAG-K-Ras 4B_{G12V} were mixed and analyzed by SEC (FIG. 11E). As a control, the proteins were individually analyzed by SEC. SEC elution profile of standard proteins is shown by a gray line with the molecular weight of peaks. Western blot analysis of SEC fractions using anti-FLAG antibodies is shown below. Purified mTORC2 using FLAG-TOR was incubated with GTP-bound FLAG-RasC_{Q62L} or FLAG-K-Ras 4B_{G12V} along with the indicated FLAG-RacE proteins in the presence of ATP and human unactive AKT (FIG. 11F). AKT phosphorylation was analyzed by Western blotting using anti-phospho AKT (serine 473) antibodies. All of the experiments were repeated at least three times.

### DETAILED DESCRIPTION

The present disclosure provides peptides for use in modulating Ras protein activity. The peptides target the inter-molecular interface created by the helix α5 of K-Ras 4B and RhoA. The helix α5 is highly conserved in Ras and Rho GTPases in Dictyostelium and human cells. Dictyostelium Ras (RasC) and Rho (termed RacE) associate through specific charged residues in the helix α5 of each GTPase. Since the phosphorylation sites for human K-Ras 4B (S181) and RhoA (S188) are located close to the helix α5, the helix α5 interaction may change upon phosphorylation.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “alkyl,” as used herein, means a straight or branched, saturated hydrocarbon chain. Representative examples of alkyl include, but are not limited to, methyl, ethyl, n-propyl, isopropyl, n-butyl, sec-butyl, iso-butyl, tent-butyl, n-pentyl, isopentyl, neopentyl, and n-hexyl.

The term “alkenyl,” as used herein, means a straight or branched, hydrocarbon chain containing at least one carbon-carbon double bond. The double bond(s) may be located at any positions with the hydrocarbon chain.

The term “alkynyl,” as used herein, means a straight or branched, hydrocarbon chain containing at least one carbon-carbon triple bond. The triple bond(s) may be located at any positions with the hydrocarbon chain.

In some instances, the number of carbon atoms in a hydrocarbyl substituent (e.g., alkyl alkenyl) is indicated by the prefix “Cₓ-C_{y},” wherein x is the minimum and y is the maximum number of carbon atoms in the substituent. Thus, for example, “C₁-C₃alkyl” refers to an alkyl substituent containing from 1 to 3 carbon atoms.

A “chemotherapeutic agent.” as used herein, refers to a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinas inhibitors. Chemotherapeutic agents include compounds used in “targeted therapy” and conventional chemotherapy. Examples of chemotherapeutic agents include, but are not limited to: erlotinib, docetaxel, 5-FU, gemcitabine, PD-0325901, cisplatin, carboplatin, paclitaxel, trastuzumab, temozolomide, doxorubicin, Akti-1/2, HPPD, and rapamycin.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

“Polynucleotide,” “oligonucleotide.” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The polypeptide can be natural, synthetic, or a modification or combination of natural and synthetic. The peptide or polypeptide may be modified by the addition of sugars, lipids or other moieties not included in the amino acid chain. The terms “polypeptide”, “oligopeptide,” and “peptide” are used interchangeably herein. The peptide(s) may be produced by recombinant genetic technology or chemical synthesis. The peptide(s) may be isolated and purified by any number of standard methods including, but not limited to, differential solubility (e.g., precipitation), centrifugation, chromatography (e.g., affinity, ion exchange, and size exclusion), or by any other standard techniques known in the art.

The recitations “sequence identity,” “percent identity,” “percent homology,” “percent similarity.” or, for example, comprising a “sequence 50% identical to” or “sequence with at least 50% similarity to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (e.g., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using an NWSgapdna.CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), sclenocysteine (present in many non-eukaryotes as well as most eukaryotes), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid. According to certain embodiments, a peptide inhibitor comprises an intramolecular bond between two amino acid residues present in the peptide inhibitor. It is understood that the amino acid residues that form the bond will be altered somewhat when bonded to each other as compared to when not bonded to each other. Reference to a particular amino acid is meant to encompass that amino acid in both its unbonded and bonded state. For example, the amino acid residue homoSerine (hSer) or homoSerine(Cl) in its unbonded form may take the form of 2-aminobutyric acid (Abu) when participating in an intramolecular bond according to the present invention.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader.

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). Unless otherwise indicated, three-letter and single-letter abbreviations of amino acids refer to the L-isomeric form of the amino acid in question. The term “L-amino acid,” as used herein, refers to the “L” isomeric form of a peptide, and conversely the term “D-amino acid” refers to the “D” isomeric form of a peptide (e.g., Dasp, (D)Asp or D-Asp; Dphe, (D)Phe or D-Phe). Amino acid residues in the D isomeric form can be substituted for any L-amino acid residue, as long as the desired function is retained by the peptide. D-amino acids may be indicated as customary in lower case when referred to using single-letter abbreviations.

In the case of less common or non-naturally occurring amino acids, unless they are referred to by their full name (e.g., sarcosine, ornithine, etc.), frequently employed three- or four-character codes are employed for residues thereof, including, Sar or Sarc (sarcosine, e.g., N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and 8Ado (8-amino-3,6-dioxaoctanoic acid), Abu (2-amino butyric acid), βhPro (β-homoproline), βhPhe (β-homophenylalanine) and Bip (β,β diphenylalanine), and Ida (Iminodiacetic acid).

The term “pharmaceutically acceptable salt” in the context of the present invention (pharmaceutically acceptable salt of a peptide described herein) refers to a salt which is not harmful to a patient or subject to which the salt in question is administered. It may suitably be a salt chosen, e.g., among acid addition salts and basic salts. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like. Other examples of pharmaceutically acceptable salts are described in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3′ edition, James Swarbrick (Ed.), Informa Healthcare USA (Inc.), NY, USA, 2007, and in J. Pharm. Sci. 66: 2 (1977).

As used herein, the terms “providing,” “administering.” and “introducing,” are used interchangeably herein and refer to the placement of the peptides or compositions of the disclosure into a subject by a method or route which results in at least partial localization to a desired site. The peptides or compositions can be administered by any appropriate route which results in delivery to a desired location in the subject.

The term “solvate” in the context of the present invention refers to a complex of defined stoichiometry formed between a solute (the peptide or pharmaceutically acceptable salt thereof described) and a solvent. The solvent in this connection may, for example, be water, ethanol, or another pharmaceutically acceptable, typically small-molecular organic species, such as, but not limited to, acetic acid or lactic acid. When the solvent in question is water, such a solvate is normally referred to as a hydrate.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species: farm animals such as cattle, horses, sheep, goats, swine: domestic animals such as rabbits, dogs, and cats: laboratory animals including rodents, such as rats, mice and guinea pigs, and the like. Examples of non-mammals include, but are not limited to, birds, fish, and the like. In one embodiment, the mammal is a human.

As used herein, “treat,” “treating,” and the like means a slowing, stopping, or reversing of progression of a disease or disorder when provided a peptide or composition described herein to an appropriate subject. The term also includes a reversing of the progression of such a disease or disorder to a point of eliminating or greatly reducing the disease. As such, “treating” means an application or administration of the peptides or compositions described herein to a subject, where the subject has a disease or a symptom of a disease, where the purpose is to cure, heal, alleviate, relieve, alter, remedy, ameliorate, improve, or affect the disease or symptoms of the disease.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Peptides and Polynucleotides

The present disclosure provides peptides, or a pharmaceutically acceptable salt or solvate thereof, comprising an amino acid sequence with at least 50% similarity (e.g., at least 55%, at least 60%, at least 65%, at least 70%, at least 75%, at least 80%, at least 85%, at least 90%, at least 95%, at least 98%, or at least 99% similarity) to SEQ ID NO: 1. In some embodiments, the peptide comprises the amino acid sequence of SEQ ID NO: 1.

SEQ ID NO: 1
GVDDAFYTLVREIRKHKEKMSKDG

In some embodiments, the peptide comprising an amino acid sequence with at least 50% similarity to SEQ ID NO: 1 comprises conservative substitutions (e.g., one or more amino acids are replaced by another, biologically similar residue defined by polarity, charge, acidity, hydrophobicity, or chemical structure (e.g., aromaticity). In some embodiments, the peptide comprising an amino acid sequence with at least 50% similarity to SEQ ID NO: 1 comprises radical substitutions (e.g., one or more amino acids are replaced by residue with different physiochemical properties defined by polarity, charge, acidity, hydrophobicity, or chemical structure (e.g., aromaticity). In some embodiments, the peptide comprises and amino acid sequence having 1, 2, 3, 4, 5, 6, 7, 8, 9, 10, 11, or 12 substitutions compared to SEQ ID NO: 1.

In some embodiments, the peptide further comprises a trafficking sequence. Trafficking sequences are amino acid sequences that direct a peptide to a desired intracellular or extracellular destination. The trafficking sequence may comprise a cell-penetrating peptide sequence, also known as a protein transduction domain, a trojan peptide, and a membrane translocating sequence. The trafficking sequence may direct or facilitate transport of the peptide across the plasma membrane and into the cytoplasm (e.g., from outside the cell to inside the cell). Alternatively, or in addition, the trafficking sequence may direct the peptide to a desired subcellular location, e.g., a particular organelle (e.g., nucleus, lysosome, etc.) or component (e.g., cellular protein).

The trafficking sequence may have a length of 5 to 150 amino acids, e.g., 5 to 100, 5 to 50, 5 to 30, or even 5 to 15 amino acids. The trafficking sequence may be continuous to the sequence of at least 50% similarity to SEQ ID NO: 1 or may be separated by additional amino acids.

The trafficking sequence may be derived from a naturally occurring source or can be based on genetic engineering. Sources for the trafficking sequence include a protein, functional fragment, or derivative thereof, including, but not limited to, the HIV-1 TAT protein, HSV-1 VP22, Antennapedia protein (Antp), Fushi tarazu (Ftz) homeodomain, Engrailed (En) homeodomain, or basic peptides having at least 80% basic, positively charged, amino acids.

In some embodiments, the trafficking sequence is derived from the HIV TAT protein. In some embodiments, the trafficking sequence comprises an amino acid sequence of RKKRRQRRR (SEQ ID NO: 8). In select embodiments, the trafficking sequence comprises the amino acid sequence of GRKKRRQRRRPQ (SEQ ID NO: 4).

In some embodiments, the peptides are modified to stabilize them, to facilitate their uptake and/or absorption, or to improve any other characteristic or property of the peptides that is known to one of skill in art. For example, the peptides can be cyclized, charges on the peptides can be neutralized, and the peptides can be linked to other chemical moieties.

Peptides can be cyclized by any method available to one of skill in the art. For example, the N-terminal and C-terminal ends can be condensed to form a peptide bond by known procedures. Functional groups present on the side chains of amino acids in the peptides can also be joined to cyclize the peptides of the invention. For example, functional groups that can form covalent bonds include —COOH and —OH; —COOH and —NH₂; and —COOH and —SH. Pairs of amino acids that can be used to cyclize a peptide include, Asp and Lys; Glu and Lys; Asp and Arg; Glu and Arg; Asp and Ser; Glu and Ser; Asp and Thr; Glu and Thr; Asp and Cys; and Glu and Cys. Other examples of amino acid residues that are capable of forming covalent linkages with one another include cysteine-like amino acids such Cys, hCys, β-methyl-Cys and Penicillamine (Pen), a non-proteinogenic an alpha-amino acid having the structure of valine substituted at the beta position with a sulfanyl group, which can form disulfide bridges with one another. Preferred cysteine-like amino acid residues include Cys and Pen. Other pairs of amino acids that can be used for cyclization of the peptide will be apparent to those skilled in the art.

The groups used to cyclize a peptide need not be amino acids. Examples of functional groups capable of forming a covalent linkage with the amino terminus of a peptide include carboxylic acids and esters. Examples of functional groups capable of forming a covalent linkage with the carboxyl terminus of a peptide include —OH, —SH, —NH₂ and —NHR where R is (C₁-C₆) alkyl, (C₁-C₆) alkenyl and (C₁-C₆) alkynyl.

The variety of reactions between two side chains with functional groups suitable for forming such interlinkages, as well as reaction conditions suitable for forming such interlinkages, will be apparent to those of skill in the art. Preferably, the reaction conditions used to cyclize the peptides are sufficiently mild so as not to degrade or otherwise damage the peptide. Suitable groups for protecting the various functionalities as necessary are well known in the art (see, e.g., Greene & Wuts, 1991, 2nd ed., John Wiley & Sons, NY), as are various reaction schemes for preparing such protected molecules.

In some embodiments, the peptides of the present invention are protease resistant. In one embodiment, such protease-resistant peptides are peptides comprising protecting groups. For example, the peptides disclosed herein may be protected from exoprotease degradation by N-terminal acetylation (“Ac”) and C-terminal amidation. Such peptides are useful for in vivo administration because of their resistance to proteolysis.

In another embodiment, the present invention also contemplates peptides protected from endoprotease degradation by the substitution of L-amino acids in the peptides with their corresponding D-isomers. It is not intended that the present invention be limited to particular amino acids and particular D-isomers. This embodiment is feasible for all amino acids, except glycine; that is to say, it is feasible for all amino acids that have two stereoisomeric forms. By convention, these mirror-image structures are called the D and L forms of the amino acid. These forms cannot be interconverted without breaking a chemical bond. With rare exceptions, only the L forms of amino acids are found in naturally occurring proteins.

The peptides may be modified to improve one or more properties useful in pharmaceutical compositions. For example, in some embodiments, the disclosed peptides are modified to enhance their ability to enter intracellular space. Such modifications include, but are not limited to, the addition of charged groups, lipids, and myristate groups (See e.g., U.S. Pat. No. 5,607,691).

The disclosed peptides may be in a liposome in which the peptide is combined with amphipathic agents such as lipids which exist in aggregated form as micelles, insoluble monolayers, liquid crystals, or lamellar layers which in aqueous solution. Suitable lipids for liposomal formulations include, without limitation, monoglycerides, diglycerides, sulfatides, lysolecithin, phospholipids, saponin, and bile acids. Preparation of such liposomal formulations is within the level of skill in the art.

The peptides may also be modified by the addition of: radioactive atoms; detectable labels (e.g., radioactive labels, dyes, fluorescent moieties, chemiluminescent moieties, quantum dots); affinity tags (e.g., His tag, biotin); PEG moieties; carbohydrates (e.g., glycosylation, hesylation); and organic molecules (e.g., alkylation, acetylation, acylation).

The present disclosure also provides for nonpeptide compounds that mimic peptide sequences (“mimetics”), synthesis of which are known in the art. Peptide mimetics that are structurally related to therapeutically useful peptides may be used to produce an equivalent or enhanced therapeutic or prophylactic effect. Generally, peptidomimetics are structurally similar to the peptide of interest, but have one or more peptide linkages optionally replaced by linkages such as —CH₂NH—, —CH₂S—, —CH₂CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such polypeptide mimetics may have significant advantages over naturally occurring polypeptides including more economical production, greater chemical stability, enhanced pharmacological properties (e.g., half-life, absorption, potency, efficiency), reduced antigenicity, and the like.

Longer peptides which result from the addition of additional amino acid residues added to the peptides described herein are also encompassed in the present invention. Such longer peptides can be expected to have the same biological activity and specificity (e.g., cell tropism) as those without the additional aminos acids. It is recognized that some large polypeptides may assume a configuration that masks the effective sequence, thereby preventing the desired activity. Confirmation of the desired activity (e.g., inhibition of K-Ras) can be collected using the assays described elsewhere herein.

The present disclosure also provides a polynucleotide encoding the peptides. In some embodiments, the polynucleotides disclosed herein can be introduced into an expression vector, such that the expression vector comprises a promoter and the polynucleotides encoding the peptides or polypeptides described herein.

The expression vector may allow expression of the peptides in a suitable expression system using techniques well known in the art, followed by isolation or purification of the expressed peptide or polypeptide of interest. The expression vector may allow expression of the peptides in an organism, in an isolated cell or cell line, or in a cell-free system. A variety of bacterial, yeast, plant, mammalian, and insect expression systems are available in the art and any such expression system can be used. Alternatively, a polynucleotide encoding a peptide of the invention can be translated in a cell-free translation system.

As such, the disclosure provides an isolated cell comprising the polynucleotide disclosed herein. Preferred cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyes, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993), incorporated herein by reference. Desirably, the cell is a mammalian cell, and in some embodiments, the cell is a human cell. A number of suitable mammalian and human cells are known in the art, and many are available from the American Type Culture Collection (ATCC, Manassas, Va.). Examples of suitable mammalian cells include, but are not limited to, Chinese hamster ovary cells (CHO), human embryonic kidney (HEK) 293 or 293T cells, 3T3 cells, monkey COS-1 and COS-7 cell lines, as well as the CV-1 cell line, mouse neuroblastoma N2A cells, HeLa, HEK, A549, HepG2, mouse L-929 cells, and BHK or HaK hamster cell lines. Further exemplary mammalian host cells include primate, rodent, and human cell lines, including transformed cell lines. Normal diploid cells, cell strains derived from in vitro culture of primary tissue, as well as primary explants, are also suitable. Methods for selecting suitable mammalian host cells and methods for transformation, culture, amplification, screening, and purification of cells are known in the art.

### 3. Compositions

Disclosed herein are compositions comprising the peptides or polynucleotides described above. The compositions may further comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate, powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil: glycols; including propylene glycol; esters including, but not limited to, ethyl oleate and ethyl laurate: agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, preservatives, and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

The compositions may be formulated for any appropriate manner of administration, and thus administered, including for example, oral, nasal, intravenous, intravaginal, epicutaneous, sublingual, intracranial, intradermal, intraperitoneal, subcutaneous, intramuscular administration, or via inhalation. Techniques and formulations may generally be found in “Remington's Pharmaceutical Sciences,” (Meade Publishing Co., Easton, Pa.). Therapeutic or pharmaceutical compositions must typically be sterile and stable under the conditions of manufacture and storage. The route or administration and the form of the composition usually dictates the type of carrier to be used.

The compositions may also comprise buffers (e.g., neutral buffered saline or phosphate buffered saline), carbohydrates (e.g., glucose, mannose, sucrose or dextrans), mannitol, antioxidants, bacteriostats, chelating agents such as EDTA or glutathione, solutes that render the formulation isotonic, hypotonic, or weakly hypertonic with the blood of a recipient, suspending agents, thickening agents and/or preservatives, commonly found in proteinaceous compositions.

### 4. Methods

The disclosed peptides, polynucleotides, and compositions may be used in various methods, including methods for treating or preventing a disease or disorder in a subject or methods for modulating the activity of a Ras protein.

In one embodiment, provided is a method for treating or preventing a disease or disorder in a subject comprising administering an effective amount of the peptides, polynucleotides, and compositions described herein to the subject. In some embodiments, the subject is a human.

The disclosed peptides of the invention are modulators of Ras protein and thus are useful in the treatment of diseases and disorders which are mediated or characterized by abnormal Ras protein activity and/or hyperactive Ras protein activity. Characterized refers herein to a disease or disorder that results, entirely or partially, from hyperactive Ras or abnormal Ras signaling (e.g., enzyme activity, protein-protein binding) or a disease or disorder wherein a particular symptom of the disease or disorder is caused, entirely or partially, by hyperactive Ras or abnormal Ras signaling. In some embodiments, the disease or disorder comprises cancer or a developmental disorder.

In some embodiments, the disease or disorder is cancer. Many different cancer types and subtypes rely on pathways mediated by Ras proteins for proliferation and tumor development thus inhibitors of Ras proteins may be used to treat a wide variety of cancers. In some embodiments, the cancer may be lung cancer, colorectal cancer, pancreatic cancer, ovarian cancer, breast cancer, or any combination thereof.

In some embodiments, the disease or disorder is a developmental disorder. Ras proteins are involved in regulation of cell proliferation, differentiation, and survival, and germline mutations or abnormal activity in Ras proteins may affect a number of developmental pathways, including, for example, the Ras-Raf-MEK-ERK pathway. The developmental disorders associated with Ras proteins share phenotypic features that include facial abnormalities, heart defects, impaired growth and development, and, in some instances, a predisposition to specific cancers. In some embodiments, the disease or disorder comprises Noonan, LEOPARD (multiple lentigines, electrocardiographic conduction abnormalities, ocular hypertelorism, pulmonic stenosis, abnormal genitalia, retardation of growth and sensorineural deafness), cardio-facio-cutaneous (CFC) and Costello syndromes.

Compounds of the present disclosure may be administered to a subject by a variety of methods. In any of the uses or methods described herein, administration may be by various routes known to those skilled in the art, including without limitation oral, inhalation, intravenous, intramuscular, subcutaneous, systemic, and/or intraperitoneal administration to a subject in need thereof. In some embodiments, the compounds or compositions as disclosed herein may be administered by parenteral administration (including, but not limited to, subcutaneous, intramuscular, intravenous, intraperitoneal, intracardiac and intraarticular injections).

The terms “effective amount” or “therapeutically effective amount,” as used herein, refer to a sufficient amount of the peptides or compositions disclosed herein being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the disclosed peptides or compositions required to provide a clinically significant decrease in disease symptoms.

The amount of the peptides or compositions of the present disclosure required for use in treatment or prevention will vary not only with the particular peptide or composition selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages of a compound of the present invention, or composition thereof, can be determined by comparing their in vitro activity, and in vivo activity in animal models.

Dosage amount and interval may be adjusted individually to provide plasma levels of the active agent which are sufficient to maintain the modulating effects, or minimal effective concentration (MEC). The MEC will vary for each peptide but can be estimated from in vivo and/or in vitro data. Dosages necessary to achieve the MEC will depend on individual characteristics and route of administration. However, bioassays can be used to determine plasma concentrations. Dosage intervals can also be determined using MEC value. Compositions or peptides should be administered using a regimen, which maintains plasma levels above the MEC for 10-90% of the time, preferably between 30-90% and most preferably between 50-90%. In cases of local administration or selective uptake, the effective local concentration of the peptide may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate, precluding toxicity. The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be also used in veterinary medicine for non-human subjects.

Peptides and compositions disclosed herein can be evaluated for efficacy and toxicity using known methods. For example, the toxicology of a particular peptide may be established by determining in vitro toxicity towards a cell line, such as a mammalian, and preferably human, cell line. The results of such studies are often predictive of toxicity in animals, such as mammals, or more specifically, humans. Alternatively, the toxicity of particular peptides in an animal model, such as mice, rats, rabbits, dogs, or monkeys, may be determined using known methods. The efficacy of a particular peptide may be established using several recognized methods, such as in vitro methods, animal models, or human clinical trials. When selecting a model to determine efficacy, the skilled artisan can be guided by the state of the art to choose an appropriate model, dose, route of administration and/or regime.

A therapeutically effective amount of a peptide disclosed herein, or compositions thereof, may be administered alone or in combination with a therapeutically effective amount of at least one additional therapeutic agent. In some embodiments, effective combination therapy is achieved with a single composition or pharmacological formulation that includes both agents, or with two distinct compositions or formulations, administered at the same time, wherein one composition includes a compound of this invention, and the other includes the second agent(s). Alternatively, in other embodiments, the therapy precedes or follows the other agent treatment by intervals ranging from minutes to months.

A wide range of second therapies may be used in conjunction with the compounds of the present disclosure. The second therapy may be a combination of a second therapeutic agent or may be a second therapy not connected to administration of another agent. Such second therapies include, but are not limited to, surgery, immunotherapy, radiotherapy, or a chemotherapeutic agent.

In another embodiment, provided is a method for modulating the activity of a Ras protein (H-Ras, N-Ras, or K-Ras) comprising contacting the Ras protein with an effective amount of the peptides or compositions described herein. The modulation may result in an increase in Ras protein activity or a decrease in Ras protein activity.

The activity may comprise, for example, GTPase activity, nucleotide (GTP or GDP) exchange (binding or release) or binding of exchange factor (e.g., guanine exchange factor), effector protein or second messenger binding, effector protein or second messenger activation, subcellular localization, phosphate release, downstream signaling component phosphorylation or dephosphorylation, and the like. In some embodiments, the activity comprises binding or complexation with RhoA. In some embodiments, the activity comprises insulin-stimulated AKT phosphorylation. In some embodiments, the activity comprises mTORC2 activation.

In some embodiments, the Ras protein is a wild-type Ras protein (e.g., K-Ras, N-Ras, H-Ras). In some embodiments, the Ras protein is a mutant Ras protein (e.g., K-Ras G12C, K-Ras G12D, K-Ras G12V, K-Ras G13C, K-Ras G13D, K-Ras V14I, T58I, K-Ras V152G, K-RasD153V, K-Ras F156I). In some embodiments, the K-Ras protein is a K-Ras protein (e.g., K-Ras 4A or K-Ras 4B). In some embodiments, the K-Ras protein is K-Ras 4B.

In some embodiments, the Ras protein is in a cell, such that contacting the Ras protein with the peptides, polynucleotides or compositions described herein comprising introducing the peptides, polynucleotides or compositions described herein into a cell comprising the targeted Ras protein. Methods for introducing into a cell are well known in the art and include, but are not limited to, chemical transfection, electroporation, microinjection, biolistic delivery via gene guns, or magnetic-assisted transfection.

In some embodiments, the cell is in an organism or host, such that introducing the peptides, polynucleotides or compositions described herein into the cell comprises administration to a subject. The method may comprise providing or administering to the subject, in vivo, or by transplantation of cells treated ex vivo with the peptides, polynucleotides or compositions as described elsewhere herein.

### 5. Examples

### Materials and Methods

Cells HEK293 and 3T3-L1 cells were grown in DMEM media (Sigma, D5796) supplemented with 10% FBS (Sigma, F2442) and 1% penicillin/streptomycin (Gibco, 15140-122). RhoA-KO and K-Ras-KO cells were generated using the GeneArt CRISPR Nuclease Vector with OFP Reporter Kit (Invitrogen, A21174) following the manufacturer's instructions. The RhoA (ACTGTGGGCACATACACCTC (SEQ ID NO: 5) for HEK293. CGGAAGAAACTGGTGATTGT (SEQ ID NO: 6) for 3T3-L1) and K-Ras (CAATGAGGGACCAGTACATG (SEQ ID NO: 7)) target sequences were cloned into the vector and transfected into WT cells. Based on OFP fluorescent signal, transfected cells were sorted in 96-well plates as single cells at the Johns Hopkins Bloomberg Flow Cytometry and Immunology Core. The knockout of RhoA and K-Ras was confirmed by DNA sequencing and Western blotting with antibodies to RhoA (Cell Signaling, 2117) and K-Ras (Invitrogen, 703345).

Plasmids and lentiviruses To express FLAG-tagged forms of mTOR, RAPTOR, RICTOR, and YFP-tagged forms of K-Ras 4B and RhoA in HEK293 cells, plasmid transfection was performed using Lipofectamine 3000 (Invitrogen, L3000015).

Lentiviruses were produced as described by Nguyen et al. and Yan et al. (Proceedings of the National Academy of Sciences of the United States of America 111, E2684-2693 (2014), and Oncogene 36, 3673-3685(2017), respectively). HEK293T cells were seeded at 1.5×10⁶ cells in a 10-cm dish and cultured for 24 h. The pHR-Sin plasmids carrying FLAG-K-Ras 4B, RhoA or GLUT4-Myc-GFP were co-transfected into HEK293T cells alongside with pHR-CMV8.2ΔR and pCMV-VSVG with Lipofectamine 3000. After 24 h, the culture medium was replaced with fresh medium. After an additional 24 h, the culture medium containing released viruses was collected. For lentiviral transduction of HEK293 and 3T3-L1 cells, cells were seeded at 1.5×10⁵ (HEK293) and 5.0×10⁴ (3T3-L1) cells/well in a 6-well plate and cultured for 24 h. Cells were then incubated with lentivirus in DMEM supplemented with 10% FBS and 8 μg/ml polybrene for 24 h.

Immunoprecipitation HEK293 cells were seeded at 5.0×10⁶ cells in a 10-cm dish and cultured for 16 h. Plasmids carrying FLAG-tagged mTOR, RAPTOR or RICTOR were transfected into cells using Lipofectamine 3000 for 16 h. Cells were harvested and homogenized in CHAPS lysis buffer (40 mM HEPES [pH 7.4], 120 mM NaCl, 0.3% CHAPS, 1 mM EDTA, 10% glycerol, 10 mM Na-pyrophosphate, 50 mM NaF, and proteinase inhibitor cocktail [Roche, 11836170001], and phosphatase inhibitors [Sigma, P0044 & P5726]) on ice for 10 min. Cell lysates were clarified by centrifugation at 4° C. and incubated with anti-FLAG agarose beads (Sigma, A2220) for 2 h at 4° C. with gentle agitation. The beads were washed three times using CHAPS lysis buffer, snap-frozen in liquid nitrogen, and stored at −80° C.

For immunoprecipitation using anti-RhoA antibodies, HEK293 cells were seeded at 5.0×10⁶ cells in a 10-cm dish and cultured for 16 h. Cells were washed with serum-free DMEM and incubated with serum-free DMEM for 5 h. Cells were then stimulated with 100 nM insulin (Gibco, 12585014) for 30 min. Cells were lysed with NP40 lysis buffer (20 mM Tris-HCl [pH 7.5], 50 mM NaCl, 1% NP40, 10% glycerol, protease inhibitor cocktail and phosphatase inhibitors) on ice for 10 min. Cell lysates were clarified by centrifugation at 4° C. and incubated with anti-RhoA antibodies (Cell Signaling, 2117) together with agarose beads coupled to protein A (Invitrogen, #10-1041) at 4° C. for 4 h. After washing with lysis buffer, bound fractions were eluted with 2×SDS-PAGE sample buffer and analyzed by SDS-PAGE and western blotting using appropriate antibodies.

Western blotting Proteins were separated using SDS-PAGE and transferred onto PVDF membranes. The antibodies were adiponectin (Cell Signaling, 2789), AKT (Cell Signaling, 9272), phospho-AKT (serine 473) (Cell Signaling, 9271), phospho-AKT substrate (Cell Signaling, 9614), TBC1D4/AS160 (Cell Signaling, 2670), FLAG (Sigma, F7425), GAPDH (Thermo Fisher, MA5-15738), GFP, GSK3 (Millipore, 05412), phospho-GSK3 (serine 9) (Cell signaling, 9322), IRS1 (Proteintech, 17509-1-AP), phospho-IRS1 (tyrosine 612) (Millipore, 09-432), K-Ras (Invitrogen, 703345), mTOR (Cell Signaling, 2983), myosin light chain 2 (Cell Signaling, 8505), phospho-myosin light chain 2 (Threonine 18/Serine 19) (Cell Signaling, 3674), RAPTOR (Cell Signaling, 2280), RhoA (Cell Signaling, 2117), phospho-RhoA (serine 188) (Abcam, ab41435), phospho-serine (Millipore, 2137995), RICTOR (Cell Signaling, 2114), tuberin/TSC2 (Cell Signaling, 4308), phospho-tuberin/TSC2 (threonine 1462) (Cell Signaling, 3617), NRAS (Invitrogen, 703435), HRAS (Proteintech, 18295-1-AP), mLST8 (Cell Signaling, 3274), and SIN1 (Cell Signaling, 12860). Immunocomplexes were visualized using fluorescent-labeled secondary antibodies, donkey anti-rabbit IgG Alexa Fluor 488 (Thermo Fisher, A21206), donkey anti-mouse IgG Alexa Fluor 488 (Thermo Fisher, A21202), and donkey anti-rabbit IgG Alexa Fluor 647 (Thermo Fisher, A31573), and detected using a Typhoon biomolecular imager (Amersham). Images were analyzed using ImageJ (NIH).

Protein purification from a Dictyostelium bioreactor Purification of human proteins from Dictyostelium cells was performed as described previously by Senoo et al. (Nat Cell Biol 21, 867-878(2019) and Cell Rep 33, 108427(2020)), with some modifications. Briefly, Dictyostelium cells were cultured in HL5 medium (1% protease peptone, 1% glucose, 0.5% yeast extract, 2.5 mM Na₂HPO₄, 2.5 mM KH₂PO₄ [pH 6.5]) on a rotary shaker at 200 rpm and 22° C. Plasmids carrying GFP- or FLAG-tagged forms of RhoA, K-Ras4A, and K-Ras 4B were introduced to Dictyostelium cells by electroporation, and stable cell lines carrying the plasmids were selected with G418. Before protein purification, cells were differentiated. To induce cell differentiation, exponentially growing cells were washed with development buffer (DB; 2 mM MgSO₄, 0.2 mM CaCl₂), 5 mM Na₂H₄PO₄, and 5 mM KH₂PO₄ [pH 6.5]), re-suspended at 2×10⁷ cells/ml, starved for 1 h, and shaken with 100 nM cAMP pulses at 6-min intervals for 4 h. Differentiated Dictyostelium cells were treated with DB containing 2 mM caffeine for 20 min at 22° C., and washed twice with ice-cold DB. Cells were shaken at 200 rpm at 22° C. and stimulated with 1 μM cAMP for 30 s unless otherwise described. Cells (1×10¹) individually expressing the human proteins were lysed in 1 ml of ice-cold NP40 lysis buffer (1% NP40, 125 mM NaCl, 1 mM EDTA, 10 mM sodium phosphate [pH 7.0], protease inhibitor cocktail [Roche 11836170001 and Sigma P8215], and phosphatase inhibitors [Sigma P5726]) for 10 min on ice. After clarification, 1 ml of cell lysate was incubated with 15 μl of GFP-Trap beads (Chromotek, gta-200) or anti-FLAG agarose beads (Sigma, A2220) to immunoprecipitate proteins for 2 h at 4° C. with gentle agitation. The beads were washed three times in NP40 lysis buffer, twice in high salt wash buffer (500 mM NaCl, 10 mM sodium phosphate [pH 7.0]), and three times in NP40 lysis buffer. The washed beads were snap-frozen in liquid nitrogen and stored at −80° C.

In vitro protein-protein interaction assay To analyze interactions between RhoA, K-Ras4A, and K-Ras 4B in vitro, purified proteins that were attached to anti-FLAG beads or GFP-Trap beads were high-salt washed with 500 mM NaCl and 50 mM HEPES (pH 7.4). To exchange guanine nucleotides that are associated with RhoA. K-Ras4A, and K-Ras 4B, purified GFP-RhoA. FLAG-K-Ras4A, and -K-Ras 4B were treated with EDTA (25 mM) for 30 min on ice, washed, and incubated with GDP (2.5 mM) or GTPγS (0.5 mM) for 30 min on ice.

FLAG-tagged K-Ras4A and K-Ras 4B were eluted from anti-FLAG beads by incubation with 1 M arginine at room temperature for 5 min. Amounts of purified proteins were quantified on CBB-stained SDS-PAGE gels using ImageJ software with BSA as a standard. After the arginine elution, FLAG-tagged K-Ras4A or K-Ras 4B proteins (250 ng) were incubated with GFP-Trap beads carrying GFP-RhoA (250 ng) in 80 μl of 1 mM MgCl2, 100 mM potassium acetate, 37.5 mM HEPES (pH 7.4) at room temperature for 15 min. GFP-Trap beads were washed three times in 1 mM MgCl2, 100 mM potassium acetate, 25 mM HEPES (pH 7.4), and bound proteins were analyzed by Western blotting.

In vitro reconstitution of mTORC2-mediated AKT phosphorylation The kinase activity of mTORC1 and mTORC2 was measured as described previously by Huang, Sarbassov dos, and Senoo (Methods Mol Biol 821, 75-86(2012), Methods Mol Biol 821, 59-74(2012), and Nat Cell Biol 21, 867-878(2019), respectively) with some modifications. Briefly, HEK293 cells (70% confluence in 10-cm dish) carrying FLAG-RAPTOR or FLAG-RICTOR were lysed with 1 ml of CHAPS lysis buffer (40 mM HEPES [pH 7.4], 120 mM NaCl, 0.3% CHAPS, 1 mM EDTA, 10% glycerol, 10 mM Na-pyrophosphate, 50 mM NaF, proteinase inhibitor cocktail, and phosphatase inhibitor cocktail). After clarification, 1 ml of cell lysates was incubated with 20 μl of anti-FLAG agarose beads (Sigma, A2220) for 2 h at 4° C. with gentle agitation. The beads were washed in 1 ml of CHAPS lysis buffer three times and re-suspended in 40 μl of CHAPS lysis buffer. Beads (10 μl) were mixed with 500 ng FLAG-tagged RhoA and/or K-Ras 4B, which were bound to anti-FLAG beads. After washing once with 25 mM HEPES buffer (pH 7.4), these beads were incubated with 40 μl of 1 M arginine at room temperature for 5 min. The eluted proteins were incubated with 250 ng unactive human AKT1 (Sigma, 14-279) in 80 μl of kinase reaction buffer (1 mM ATP, 1 mM MgCl₂, 100 mM potassium acetate, and 37.5 mM HEPES [pH 7.4]) at 37° C. for 15 min with gentle mixing. Reactions were stopped by adding 80 μl of 2×SDS-PAGE sample buffer. AKT phosphorylation was detected by Western blotting with anti-phospho-AKT (serine 473) antibodies (Cell Signaling, 9271).

Insulin-induced AKT phosphorylation in cells HEK293 cells and differentiated 3T3-L1 adipocytes were washed with serum-free DMEM and incubated with serum-free DMEM for 5 h. Cells were then stimulated with 100 nM insulin (Gibco, 12585014) for 30 min. Cells were lysed with NP40 lysis buffer (20 mM Tris-HCl [pH 7.5], 50 mM NaCl, 1% NP40, 10% glycerol, protease inhibitor cocktail and phosphatase inhibitors). After clarification, cell lysates were analyzed by Western blotting.

Peptides K-tetracosapeptide (GVDDAFYTLVREIRKHKEKMSKDG (SEQ ID NO: 1)), scrambled (HRATGFLEKRKDVKVGEDSYMDIK (SEQ ID NO: 2)), TAT-fused K-tetracosapeptide (GRKKRRQRRRPQGVDDAFYTLVREIRKHKEKMSKDG (SEQ ID NO: 3)), and TAT peptides (GRKKRRQRRRPQ (SEQ ID NO: 4)) were synthesized at Biomatik (Wilmington, DE, USA).

Inhibition of protein kinases HEK293 cells were seeded at 5.0×10⁵ cells in a 12-well chamber and cultured for 16 h. Plasmids carrying YFP-RhoA were transfected into cells using Lipofectamine 3000 for 16 h. HEK293 cells stably harboring FLAG-K-Ras 4B were seeded at 5.0×10⁵ cells in a 12-well chamber and cultured for 16 h. Cells were washed with serum-free DMEM and incubated with serum-free DMEM for 5 h. Cells were treated with kinase inhibitors for 30 min and then stimulated with 100 nM insulin for 30 min. Cells were lysed with NP40 lysis buffer (20 mM Tris-HCl [pH 7.5], 50 mM NaCl, 1% NP40, 10% glycerol, protease inhibitor cocktail and phosphatase inhibitors) on ice for 10 min. Cell lysates were clarified by centrifugation at 4° C. and incubated with anti-FLAG agarose (for K-Ras 4B) or GFP trap (for RhoA) beads for 2 h at 4° C. with gentle agitation. The beads were washed three times using lysis buffer. To detect phosphorylation of K-Ras 4B at S181, FLAG-K-Ras 4B was eluted from the anti-FLAG beads with 1 M arginine and analyzed by Western blotting with antibodies to K-Ras (Invitrogen, 703345) and phospho-serine (Millipore, 2137995). Phosphorylation of RhoA at S188 was analyzed by Western blotting with antibodies to RhoA (Cell Signaling, 2117) and phospho-RhoA (S188) (Abcam, ab41435). 10 μM H-89 (Sigma, 371962), 10 μM Rp-8-bromo-cyclic AMPS (Cayman chemical, 21584), 30 nM bisindolylmaleimide IX (Cayman chemical, 13334), 3 μM BX-795 (Sigma, SML0694), 10 μM U0126 (Sigma, U120), 10 μM afuresertib (Cayman chemical, 17988), 100 nM rapamycin (Selleckchem, S1039), 50 nM LY2090314 (Selleckchem, S7063), 100 μM SQ22536 (Sigma, S153), or 30 μM KH7 (Cayman chemical, 13243) were used.

In vitro phosphorylation of K-Ras 4B and RhoA by PKA Anti-FLAG beads carrying FLAG-K-Ras 4B or FLAG-RhoA were mixed with 1000 units of the catalytic subunit of PKA (New England BioLabs, P6000S) in NEBuffer for protein kinase (50 mM Tris-HCl [pH 7.5], 10 mM MgCl₂, 0.1 mM EDTA, 2 mM DTT, 0.01% Brij 35) in the presence or absence of 200 μM ATP for 30 min at 30° C. according to the manufactures' instruction. To detect phosphorylation of K-Ras 4B at S181, FLAG-K-Ras 4B was eluted from the anti-FLAG beads with 1 M arginine and analyzed by Western blotting with antibodies to K-Ras (Invitrogen, 703345) and phospho-serine (Millipore, 2137995). Phosphorylation of RhoA at S188 was analyzed by Western blotting with antibodies to RhoA (Cell Signaling, 2117) and phospho-RhoA (S188) (Abcam, ab41435).

Differentiation of 3T3-L1 pre-adipocytes into adipocytes Differentiation of 3T3-L1 pre-adipocytes into adipocytes was performed as described (Bogan et al., Mol Cell Biol 21, 4785-4806 (2001); Reed and Lane. Proc Natl Acad Sci USA 77, 285-289(1980)). Cell culture plates were treated with 50 μg/ml of collagen I for 2 h (Thermo Fisher, A1048301). Cells were grown to reach 100% confluence for at least 2 days prior to the induction of differentiation. Differentiation was induced with DMEM culture medium supplemented with 10% FBS, 0.25 μM dexamethasone (Sigma, D4902), 1 μg/ml of insulin (Gibco, 12585014), 500 μM methylisobutylxanthine (Sigma, 15879), and 1% penicillin/streptomycin. After 48 h, the culture medium was replaced by DMEM medium supplemented with 10% FBS, 1% penicillin/streptomycin, and 1 μg/ml insulin. After another 48 h. the cells were cultured in DMEM medium with 10% FBS and 1% penicillin/streptomycin for 2-4 days (the culture medium was changed every 2 days).

Oil red O stain was performed as described (Jefcoate et al, Methods Mol Biol 456, 173-193(2008)). Oil red O solution was made by mixing oil red O (Sigma, O-0625) and distilled water at 3:2 and filtered using a 22-μm filter (Millipore, SLGP033RB) to remove precipitates. Undifferentiated and differentiated 3T3-L1 cells were chemically fixed with 10% formalin neutral buffered for 30 min at room temperature. After fixation, the cells were washed once with distilled water, then with 60% isopropanol, and stained with oil red O solution for 15 min at room temperature with mild agitation. The cells were washed five times with distilled water for 5 min. Images were captured with Olympus BX51TF with DP70 color camera.

Insulin-induced AKT phosphorylation and translocation of GLUT4 in 3T3-L1 adipocytes To examine AKT phosphorylation, differentiated WT 3T3-L1 adipocytes were incubated in serum-free DMEM medium for 5 h and stimulated with 100 nM insulin for 30 min. For peptide inhibition, TAT-fused K-tetracosapeptide or TAT peptides were included in the DMEM medium for 5 h. Cells were lysed with NP40 lysis buffer (20 mM Tris-HCl [pH 7.5], 50 mM NaCl, 1% NP40, 10% glycerol, protease inhibitor cocktail [Roche, 11836170001] and phosphatase inhibitors [Sigma. P0044 & P5726]). After clarification, cell lysates were analyzed by Western blotting using antibodies to AKT (Cell Signaling, 9272) and phospho-AKT (Serine 473) (Cell Signaling, 9271). To analyze the intracellular localization of GLUT4, WT 3T3-L1 pre-adipocytes were transduced with lentiviruses expressing GLUT4-GFP and differentiated into adipocytes. Adipocytes were serum-starved for 5 h in the presence or absence of peptides and stimulated with insulin. Adipocytes were fixed with 4% paraformaldehyde for 20 min at room temperature, washed twice with PBS and submerged in an anti-fade reagent (Cell Signaling, 8961). Cells were viewed with Enhanced 3i Marianis/Yokogawa Spinning Disk Confocal microscopy.

### Example 1

### Insulin Promotes the Formation of KARATE, a Supercomplex Consisting of mTORC2, GTP-Bound K-Ras 4B and GDP-Bound RhoA

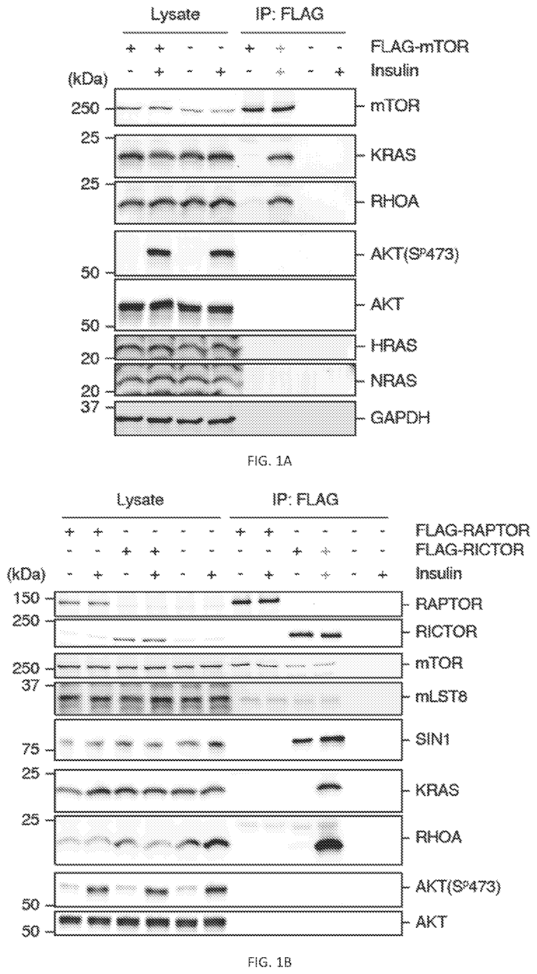

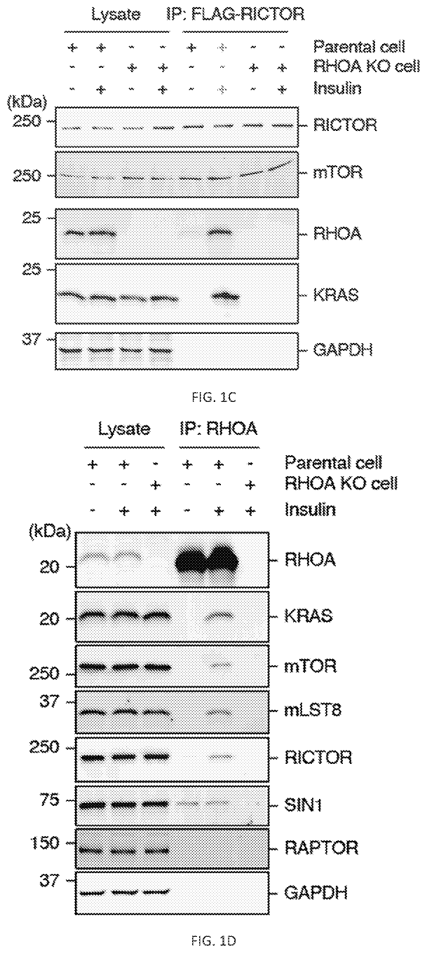

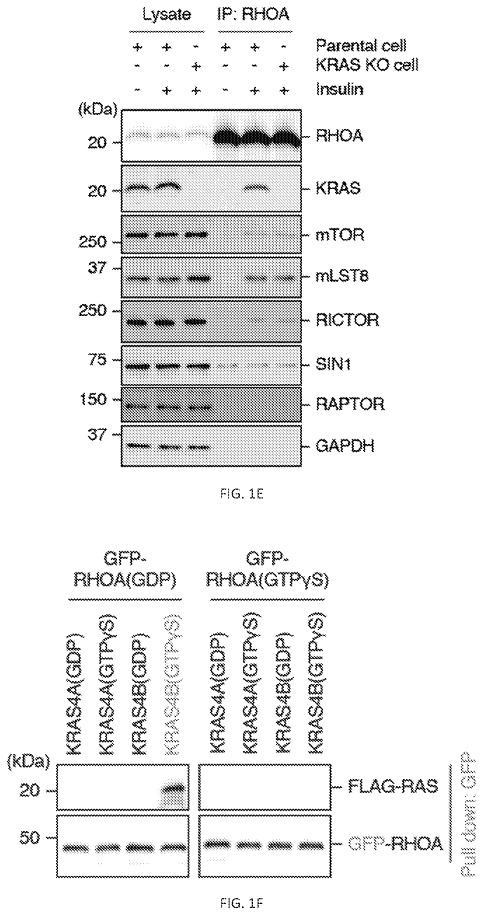

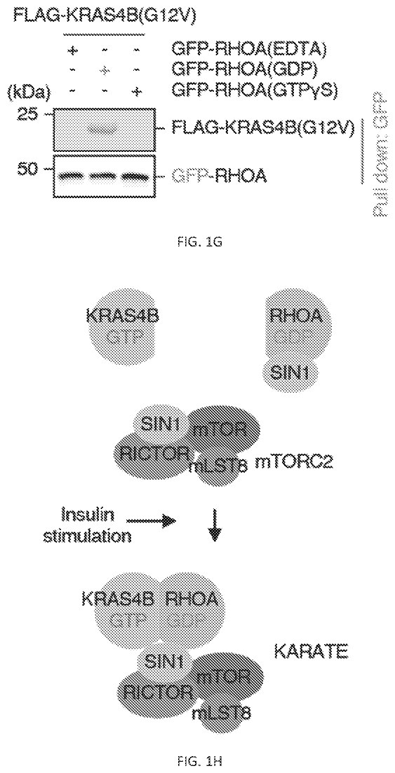

RhoA is a human homolog of Dictyostelium RacE. To test whether RhoA binds mTOR and Ras in the insulin signaling pathway, co-immunoprecipitation using FLAG-mTOR in human HEK293 cells was performed. FLAG-mTOR co-immunoprecipitated K-Ras and RhoA only when cells are treated with insulin (FIG. 1A). This interaction was specific to K-Ras since H-Ras and N-Ras were not co-immunoprecipitated. The mTOR kinase forms two functionally distinct complexes, mTORC1 and mTORC2. To identify which mTOR complex binds to K-Ras and RhoA, co-immunoprecipitation using a FLAG-tagged mTORC1-specific subunit (FLAG-RAPTOR) and a mTORC2-specific subunit (FLAG-RICTOR) was performed after insulin stimulation in HEK293 cells. FLAG-RICTOR, but not FLAG-RAPTOR, co-immunoprecipitated K-Ras and RhoA (FIG. 1B).

In Dictyostelium cells, Rho bridges Ras and mTORC2. To examine the role of RhoA in the interaction of K-Ras and mTORC2, RhoA-KO HEK293 cells were generated using CRISPR. FLAG-RICTOR was immunoprecipitated in RhoA-KO HEK293 cells and K-Ras was not co-immunoprecipitated (FIG. 1C). Therefore, RhoA connected K-Ras and mTORC2 in response to insulin. Immunoprecipitation of endogenous RhoA revealed that RhoA was associated with one of the mTORC2 subunits, SIN1, in the absence of insulin stimulation (FIG. 1D). Upon insulin stimulation, RhoA co-precipitated mTORC2 and K-Ras (FIG. 1D). These data suggested that SIN1 binds RhoA in a stimulation-independent manner and, upon insulin stimulation, SIN1 connects RhoA with mTORC2 (FIG. 1H). The loss of K-Ras affected neither RhoA-SIN1 association without insulin stimulation nor RhoA-mTORC2 association after stimulation (FIG. 1E). Therefore, it appeared that RhoA and SIN1 play important roles in the interaction of K-Ras, RhoA, and mTORC2 in response to insulin stimulation.

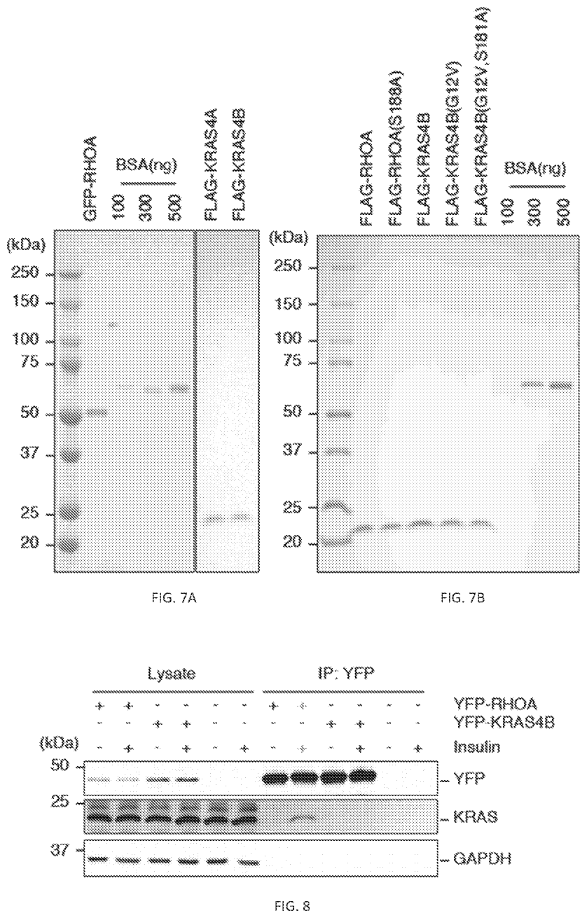

There are two isoforms of K-Ras: K-Ras 4A and K-Ras 4B. To analyze the specificity of the K-Ras isoforms, in vitro binding assays were performed using human RhoA and K-Ras proteins purified from heterologous protein expression system using Dictyostelium cells after chemotactic cAMP stimulation (FIG. 7). Purified GFP-RhoA was loaded with either GDP or GTPγS (a non-hydrolyzable GTP analog) and mixed with one of two K-Ras isoforms (K-Ras4A and K-Ras 4B), both of which were also loaded with GDP or GTPγS. GFP-RhoA was pulled down using GFP-Trap beads. Only a specific combination of GDP-RhoA and GTPγS—K-Ras 4B showed direct interaction (FIG. 1F). This guanine nucleotide code for the K-Ras 4B-RhoA interaction was confirmed using a constitutively GTP-bound K-Ras 4B_{G12V} mutant (FIG. 1G). Taken together, these data indicated that insulin induces the assembly of a supercomplex that consists of K-Ras 4B, RhoA, and mTORC2. This supercomplex is referred to herein as KARATE (K-Ras 4B-RhoA-mTORC2 Ensemble) (FIG. 1H).

### Example 2

### Purified KARATE Phosphorylates AKT In Vitro

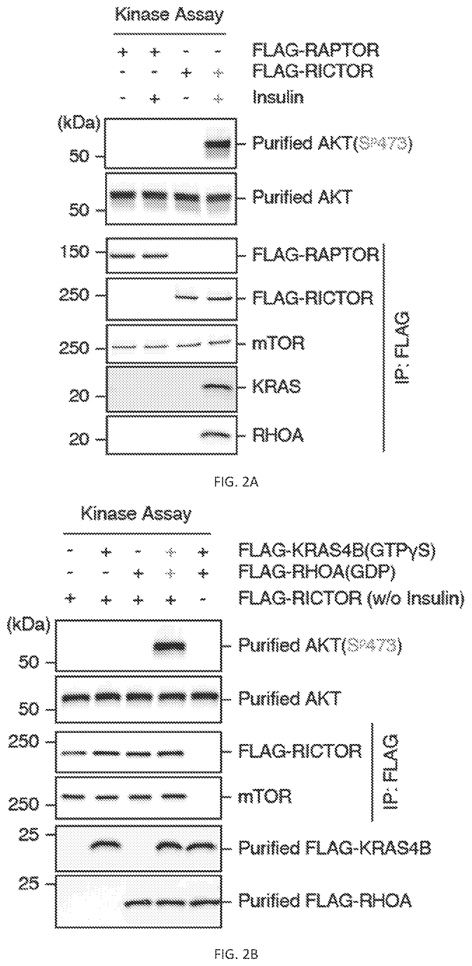

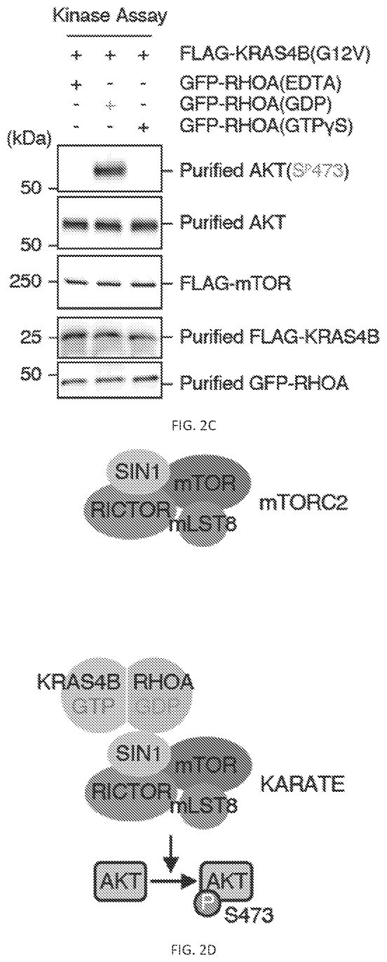

To test the role of KARATE in AKT phosphorylation, the supercomplex was immunopurified from HEK293 cells using FLAG-RICTOR after insulin stimulation, which induced the association of mTORC2 with K-Ras 4B and RhoA (FIGS. 1A-1C). The purified KARATE was incubated with recombinant AKT. KARATE phosphorylated AKT at serine 473 in vitro (FIG. 2A). Purified mTORC2 without insulin stimulation, not associated with RhoA or K-Ras 4B, failed to phosphorylate AKT (FIG. 2A). Unlike FLAG-RICTOR, the immunoprecipitant of FLAG-RAPTOR did not phosphorylate AKT (FIG. 2A).

Next, mTORC2 with FLAG-RICTOR were purified without stimulation (as described above, this mTORC2 is free from K-Ras 4B and RhoA, and does not phosphorylate AKT). Individually or in combination, purified K-Ras 4B and RhoA was added to the mTORC2. mTORC2 phosphorylated AKT only when both GTP-K-Ras 4B and GDP-RhoA were present (FIG. 2B). Furthermore, a constitutively GTP-bound K-Ras 4B G2V mutant along with GDP-loaded RhoA, but not EDTA-treated GTP/GDP-free RhoA or GTPγS-loaded RhoA, also activated mTORC2-mediated AKT phosphorylation (FIG. 2C). These data indicated that the specific combination of GTP-K-Ras 4B and GDP-RhoA is necessary for the formation of KARATE and AKT phosphorylation (FIG. 2D).

### Example 3

### K-Ras 4B-RhoA Interaction Facilitates the Formation and Activity of KARATE

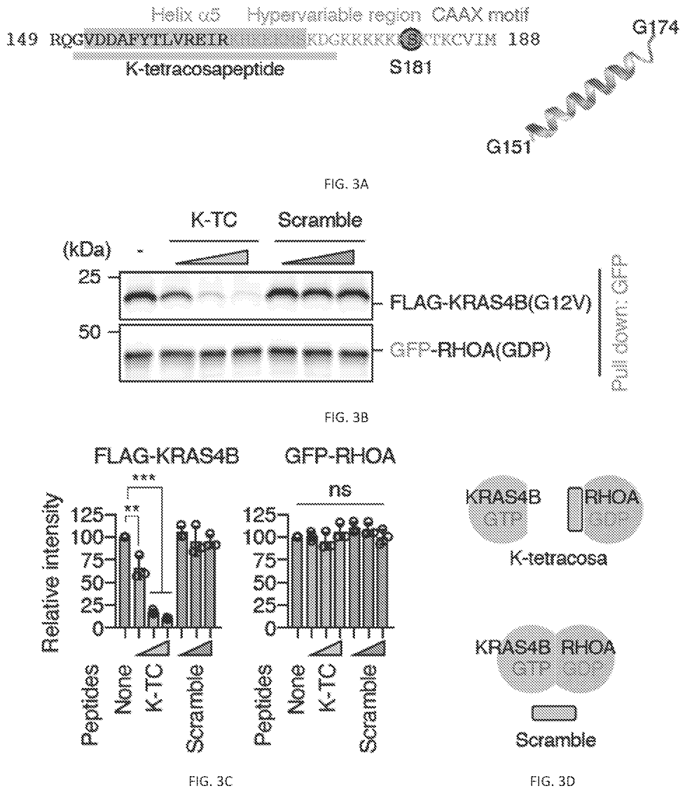

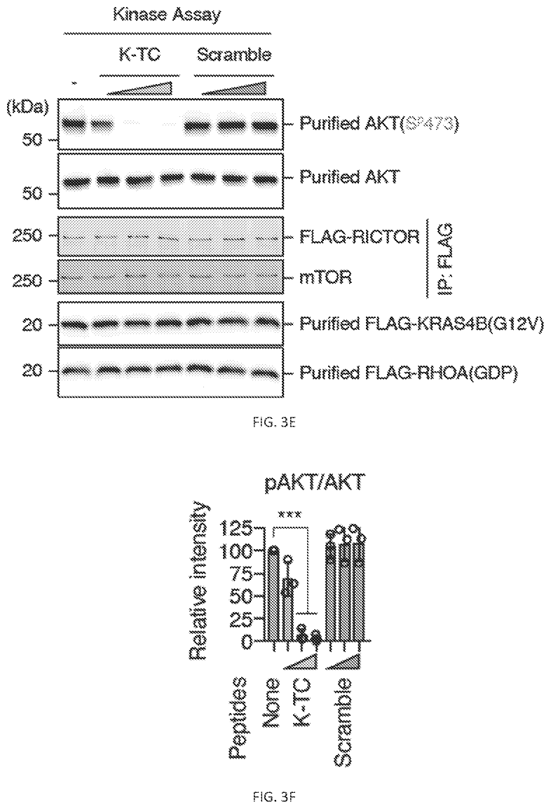

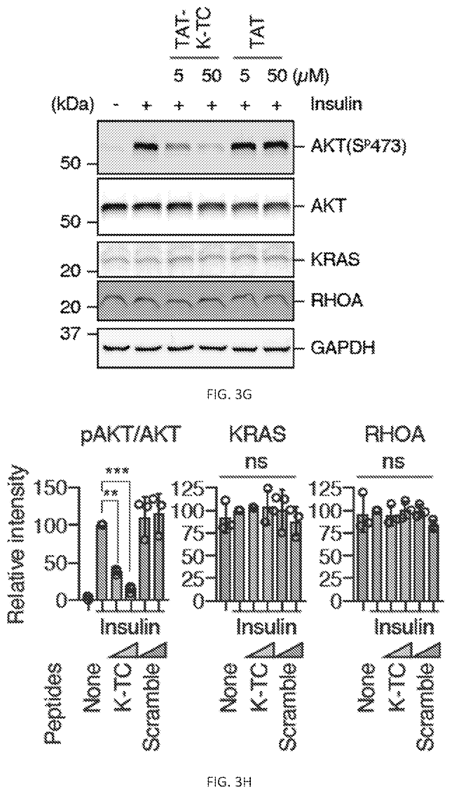

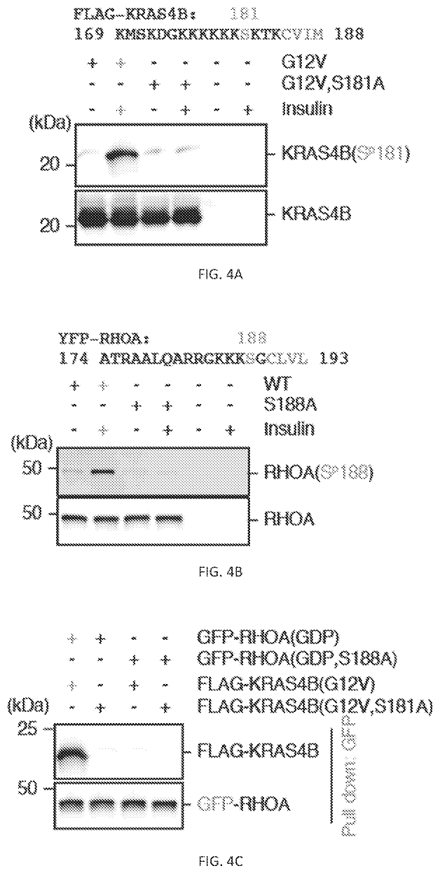

Dictyostelium Ras and Rho directly associate through the molecular interface that is formed by evolutionarily conserved helix α5 in each protein (FIG. 3A). To ask if K-Ras 4B and RhoA use the equivalent interface, a peptide of 24 amino acids corresponding to the helix α5 in K-Ras 4B (referred to as K-tetracosapeptide) was synthesized (FIG. 3A) and its effect on the K-Ras 4B-RhoA interaction was tested in vitro. K-tetracosapeptide, but not a scrambled peptide, greatly decreased the interaction of purified GTP-K-Ras 4B and GDP-RhoA in a dose-dependent manner (FIGS. 3B-3D). K-tetracosapeptide also blocked AKT phosphorylation in vitro (FIGS. 3E and F). Furthermore, when cells were incubated with K-tetracosapeptide fused to a cell-permeable TAT peptide, the insulin-stimulated AKT phosphorylation was blocked in a dose-dependent manner (FIGS. 3G and 3H). Therefore. K-tetracosapeptide was a potent inhibitor of KARATE in vitro and in cells.

Since previous studies have reported that Ras GTPases homo-dimerize via the helix α5 or other regions in oncogenic signaling, whether K-Ras 4B homo-dimerizes upon insulin stimulation was tested. K-Ras 4B bound RhoA, but not K-Ras itself, in response to insulin (FIG. 8). These data showed that K-tetracosapeptide targets the K-Ras 4B-RhoA interaction.

### Example 4

### Protein Kinase a Phosphorylates K-Ras 4B and RhoA Upon Insulin Stimulation to Promote the Formation of KARATE

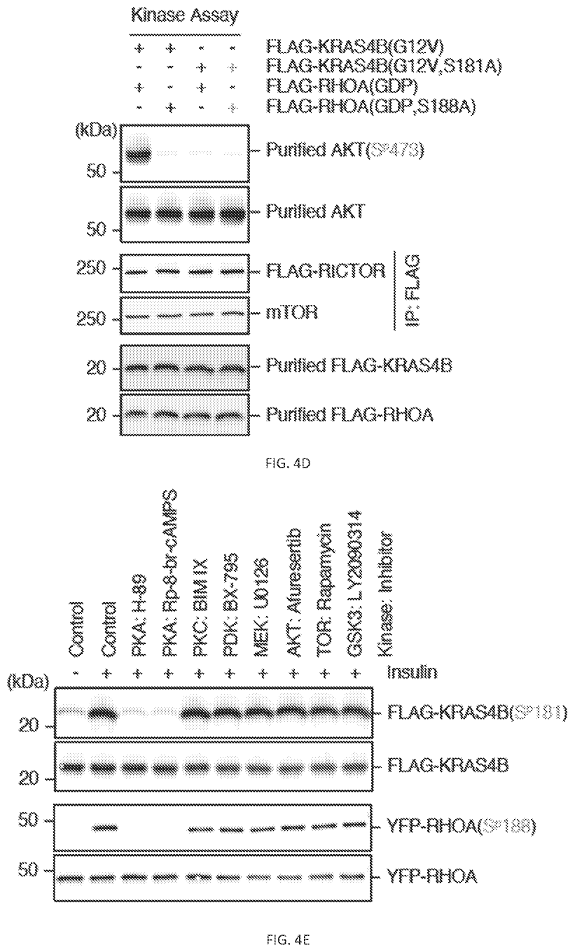

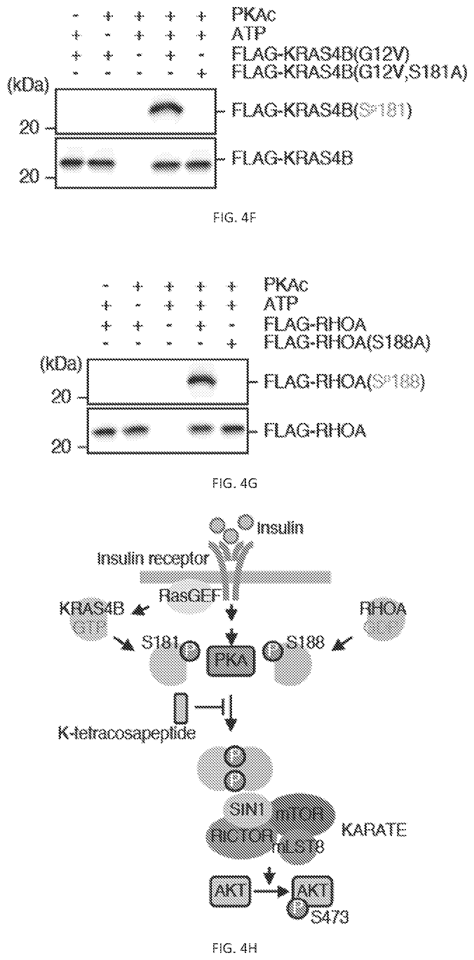

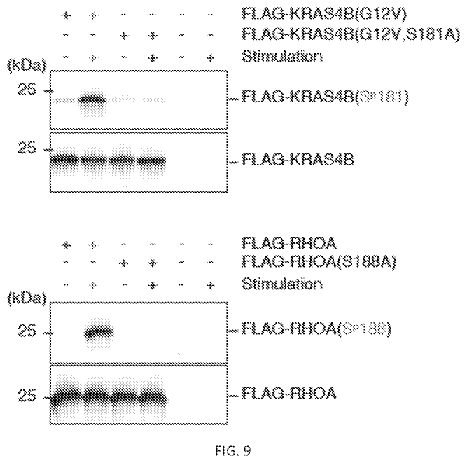

In Dictyostelium cells, the RhoA homolog RacE becomes phosphorylated at the C-terminus upon cAMP stimulation, and this phosphorylation promotes RacE-RasC interaction. To understand how insulin promotes the assembly of KARATE in human cells, the phosphorylation of K-Ras 4B and RhoA was examined. K-Ras 4B and RhoA became phosphorylated at serine 181 and 188, respectively, upon insulin stimulation (FIGS. 4A and 4B). K-Ras 4B and RhoA that were purified from Dictyostelium cells after chemoattractant stimulation were also phosphorylated at the same serine residues (FIG. 9). Phospho-defective mutations, S181A in K-Ras 4B and S188A in RhoA, blocked their interaction in vitro (FIG. 4C). Furthermore, these mutations also inhibited mTORC2-mediated AKT phosphorylation in vitro (FIG. 4D). These data showed that the insulin-induced phosphorylations of K-Ras 4B and RhoA control the assembly of KARATE.

To identify the protein kinases that phosphorylate K-Ras 4B and RhoA in response to insulin, a panel of specific inhibitors to multiple kinases was tested (PKA, PKC, PDK, MEK, AKT, TOR, and GSK3) and two structurally distinct inhibitors to PKA (protein kinase A) blocked the insulin-induced phosphorylation of both K-Ras 4B and RhoA (FIG. 4E). To determine whether these phosphorylations are direct, K-Ras 4B and RhoA were purified from Dictyostelium cells without chemoattractant stimulation, which produced unphosphorylated forms of K-Ras 4B and RhoA (FIG. 9), and these proteins were used in an in vitro kinase assay. Purified catalytic subunit of PKA phosphorylated K-Ras 4B at S181 and RhoA at S188 in vitro (FIGS. 4F and 4G). Therefore, insulin induced phosphorylation of K-Ras 4B and RhoA by PKA and assembled KARATE (FIG. 4H).

### Example 5

### K-Ras 4B and RhoA for Insulin-Induced, mTORC2-Mediated AKT Phosphorylation in Cells

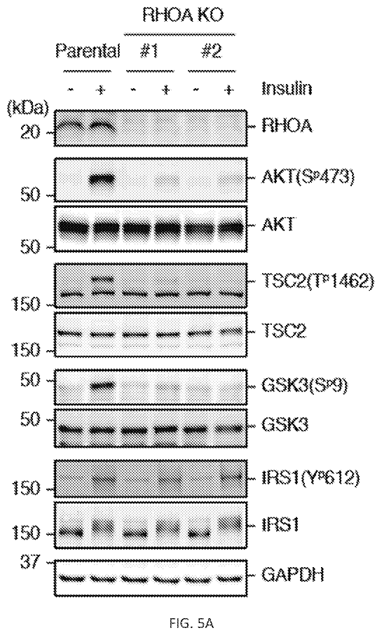

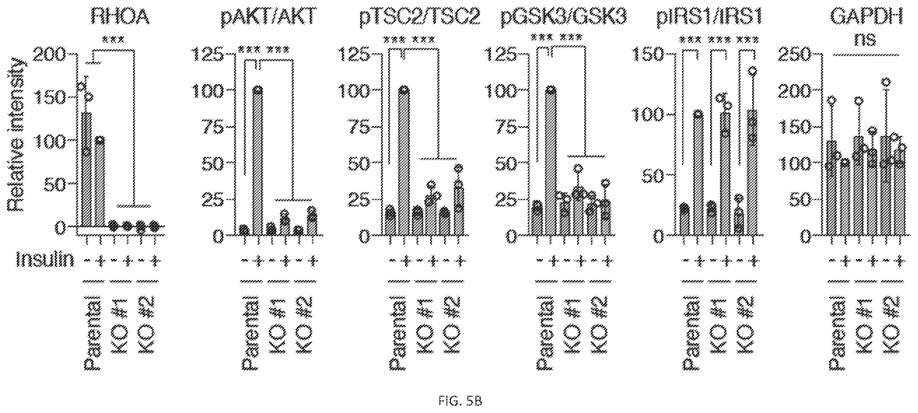

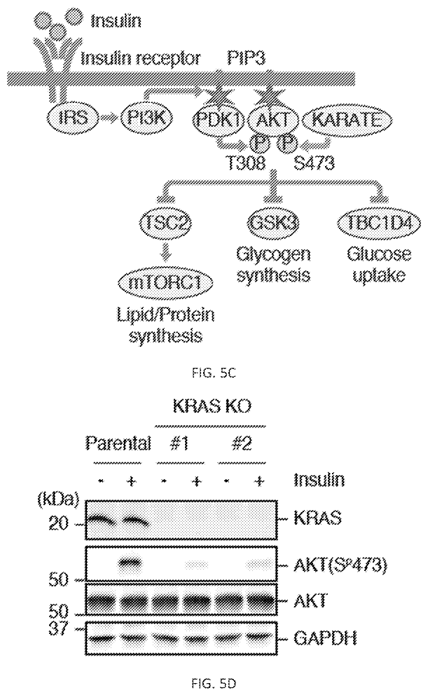

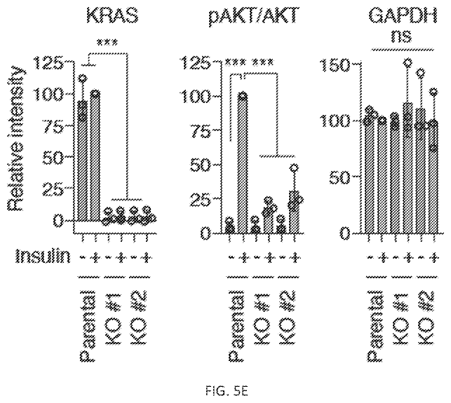

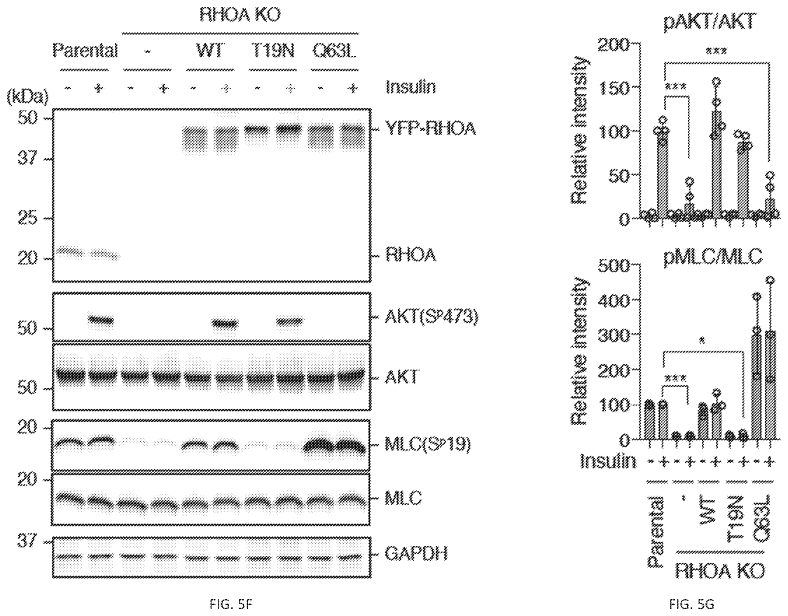

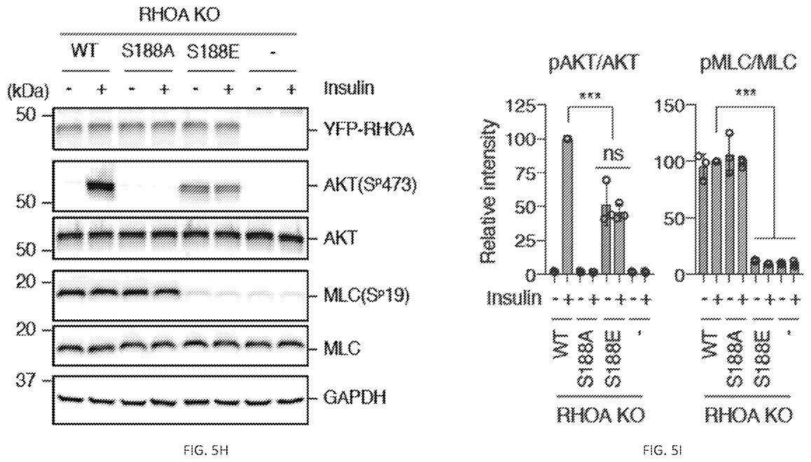

To further test the role of K-Ras and RhoA in insulin signaling in cells, insulin-stimulated AKT phosphorylation was analyzed in RhoA-KO and K-Ras-KO HEK293 cells. In parental WT cells, insulin-stimulated phosphorylation of AKT and its downstream effectors, GSK3 and TSC2 were increased (FIGS. 5A-5C). In contrast, these phosphorylations were blocked in RhoA-KO cells (FIGS. 5A-5C). The phosphorylation of insulin receptor substrate 1 (IRS1), which is immediately downstream of the insulin receptor and upstream of mTORC2, was not decreased in RhoA-KO cells (FIGS. 5A-5C). Like RhoA-KO cells, K-Ras-KO cells were unable to induce AKT phosphorylation upon insulin stimulation (FIGS. 5D and 5E). Therefore, RhoA and K-Ras directly influenced insulin-dependent AKT phosphorylation in cells. Re-expression of WT RhoA in RhoA-KO cells restored insulin-induced AKT phosphorylation (FIGS. 5F and 5G). Similarly, a constitutively GDP-bound RhoA_{T19N} rescued the AKT phosphorylation defect. In contrast, a constitutively GTP-bound RhoA_{Q63L} failed to do so (FIGS. 5F and G). These data demonstrated that GDP-bound RhoA mediated the phosphorylation of AKT in insulin signaling in cells.

To understand the role of S188-phosphorylation of RhoA in cells, WT, phospho-defective, or phospho-mimetic of RhoA were expressed in RhoA-KO cells (FIGS. 5H and 5I). While WT RhoA restored insulin-dependent phosphorylation of AKT, phospho-defective RhoA_{S188A} failed to do so. Importantly, phospho-mimetic RhoA_{S188E} led to the constitutive phosphorylation of AKT, even in the absence of insulin (FIGS. 5H and 5I). These data indicated that S188-phosphorylation of RhoA mediated AKT phosphorylation upon insulin stimulation in cells.

### Example 6

### S188-Phosphorylated GDP-Bound RhoA does not Activate ROCK1

A GTP-bound form of RhoA is known to activate a serine/threonine kinase, ROCK1, which in turn, phosphorylates myosin light chain (MLC) to control the actin cytoskeleton independently of insulin (FIGS. 5F and 5G). MLC phosphorylation was lost in RhoA-KO cells (FIGS. 5F and 5G). However, in sharp contrast to AKT, the defect of MLC phosphorylation was rescued by WT, GTP-bound RhoA_{Q63L}, phospho-defective RhoA_{S188A}, but not GDP-bound RhoA_{T19N} or phospho-mimetic RhoA_{S188E} (FIGS. 5F-5I). These data showed that RhoA is regulated via distinct mechanisms in the mTORC2-AKT pathway (activated by S188-phosphorylation and GDP-binding) and the ROCK1 pathway (S188-unphosphorylated and GTP-binding).

### Example 7

### KARATE Mediated Adipocyte Differentiation

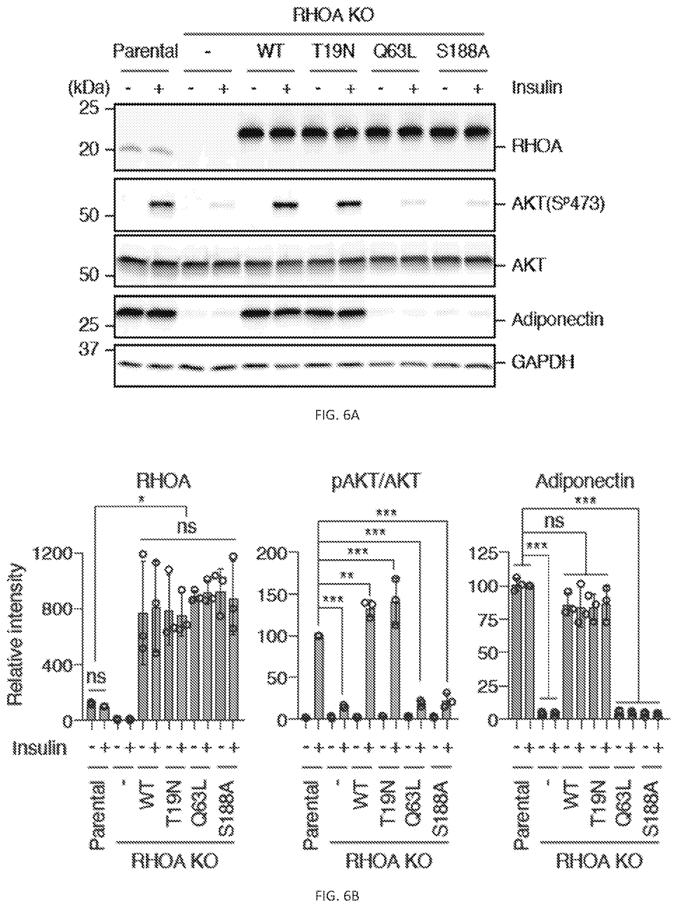

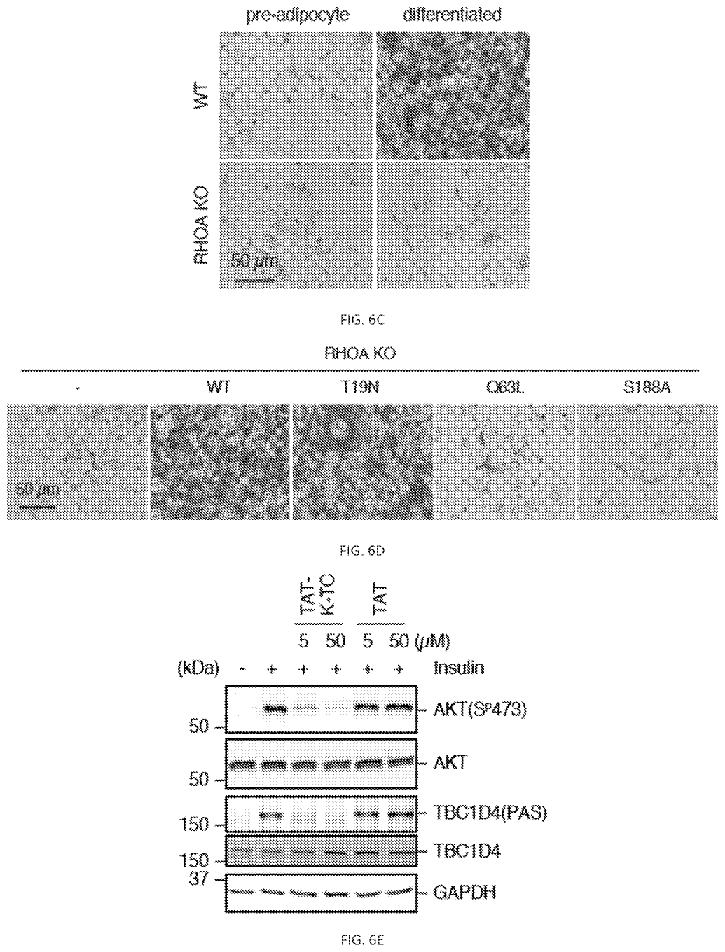

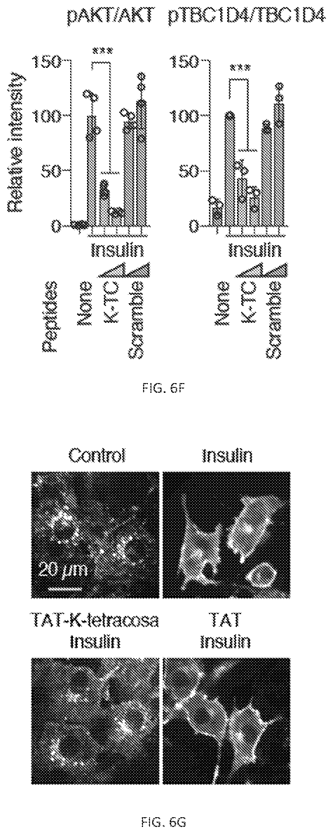

Differentiation of the pre-adipocyte mouse cell line 3T3-L1 into adipocytes depends on insulin and AKT. To test the physiological role of the RhoA, RhoA-KO 3T3-L1 cells were created using CRISPR and differentiation was induced. RhoA-KO cells failed to differentiate into adipocytes, as shown by the lack of both adiponectin expression in Western blotting (FIGS. 6A and 6B) and lipid droplets by oil red O staining (FIG. 6C). When WT, constitutively GDP-bound RhoA_{T19N}, constitutively GTP-bound RhoA_{Q63L}, or phospho-defective RhoA_{S188A} were introduced in RhoA-KO cells, WT and GDP-bound RhoA_{T19N}, but not GTP-bound RhoA_{Q63L} or phospho-defective RhoA_{S188A}, rescued the differentiation defects (FIGS. 6A, 6B and 6D). Therefore, these data suggested that the differentiation of 3T3-L1 cells into adipocytes is regulated by KARATE.

### Example 8

### KARATE Regulated the Transport of the Glucose Transporter GLUT4 in Response to Insulin

Fully differentiated WT 3T3-L1 adipocytes were stimulated with insulin after pre-treatment with TAT-K-tetracosapeptide or a TAT peptide. TAT-K-tetracosapeptide blocked insulin-induced phosphorylation of AKT and its downstream effector TBC1D4, a Rab GTPase-activating protein (FIGS. 6E and 6F). Since the phosphorylation of TBC1D4 drives the translocation of the glucose transporter GLUT4 to the plasma membrane from intracellular vesicles for glucose uptake into adipocytes, the intracellular localization of GLUT4-GFP was examined in 3T3-L1 adipocytes. TAT-K-tetracosapeptide strongly inhibited insulin-induced transport of GLUT4-GFP to the plasma membrane (FIG. 6G). Therefore, KARATE regulated insulin-induced AKT phosphorylation and GLUT4 transport in 3T3-L1 adipocytes.

### Example 9

### Dictyostelium RasC is Functionally Related to K-Ras 4B

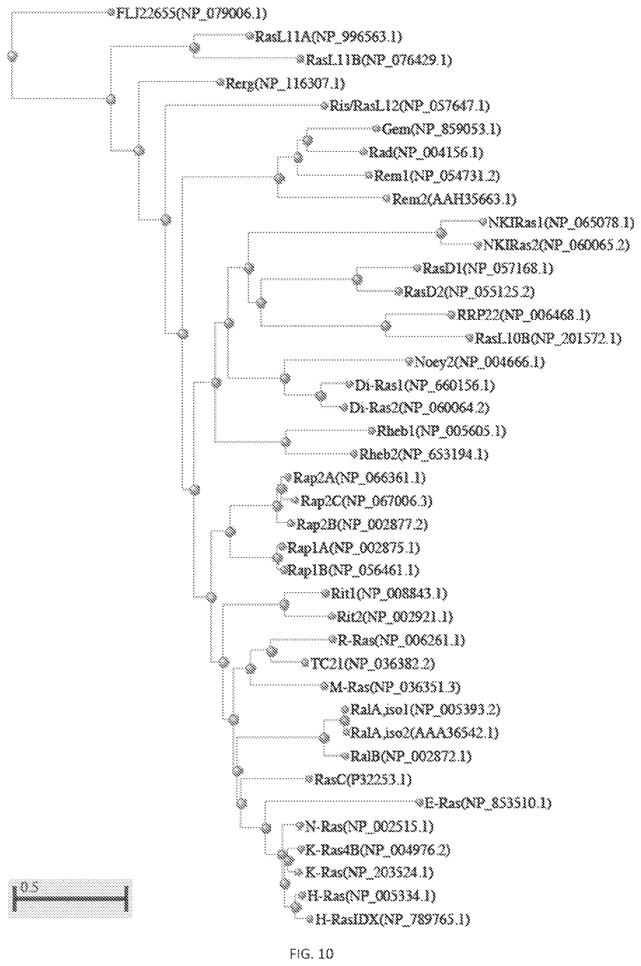

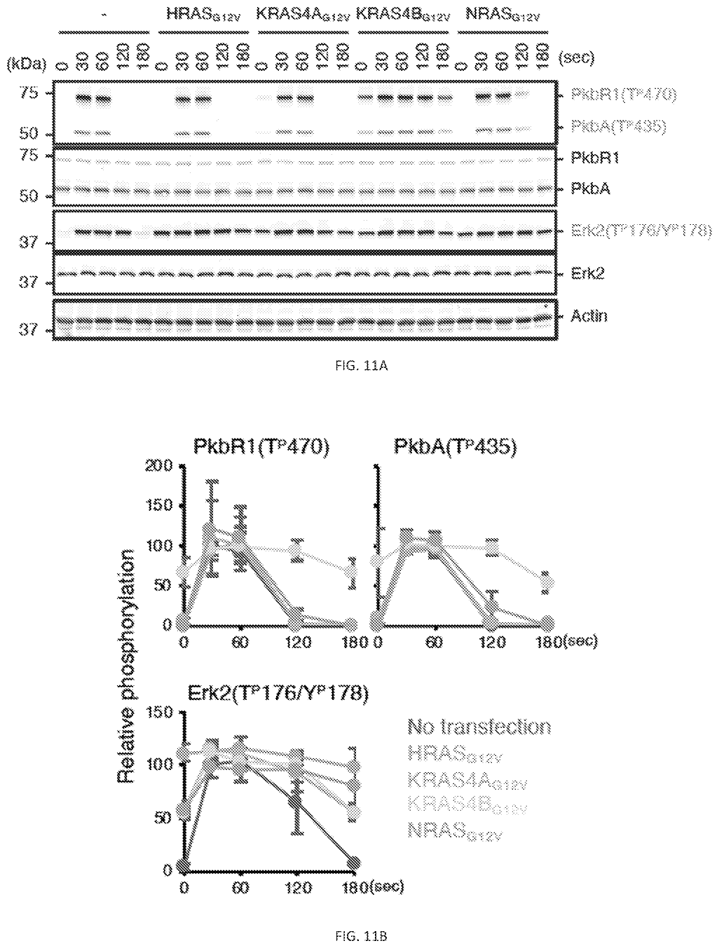

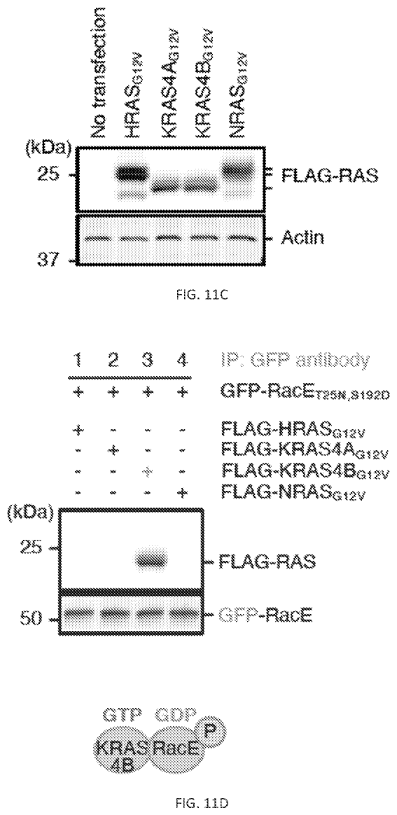

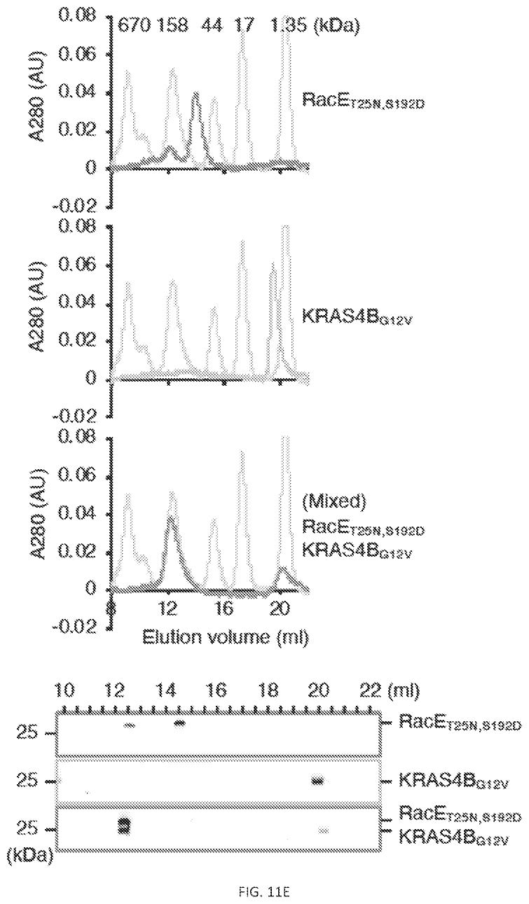

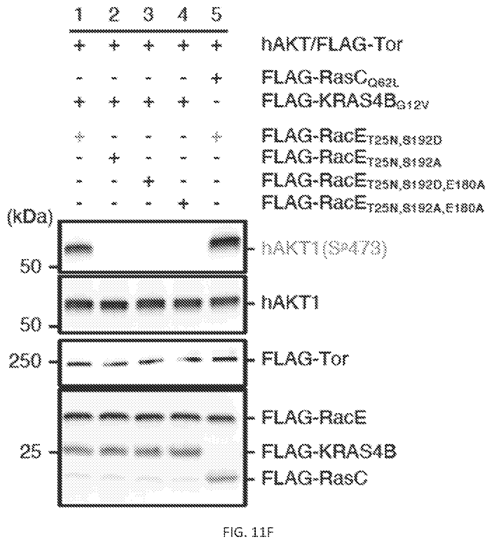

A phylogenetic analysis suggested that Dictyostelium RasC is related human H-Ras, K-Ras, and N-Ras (FIG. 10). To determine which human Ras is a functional counterpart of RasC, oncogenic, GTP-bound forms of human H-Ras_{G12V}, two isoforms of K-Ras_{G12V} (K-Ras 4A_{G12V} and K-Ras 4B_{G12V}) and N-Ras_{G12V}, were individually expressed in Dictyostelium cells and their effect on AKT phosphorylation was tested. GTP-K-Ras 4B_{G12V}, but not others, increased AKT phosphorylation, similar to GTP-RasC_{Q62L} (FIGS. 11A and 11B). The expression levels of these Ras proteins were comparable to each other (FIG. 11C). To determine if human GTP-K-Ras 4B binds to Dictyostelium RacE, FLAG-H-Ras_{G12V}, FLAG-K-Ras 4A_{G12V}, FLAG-K-Ras 4B_{G12V}, and FLAG-N-Ras_{G12V} were purified and incubated with purified phosphomimetic GDP-RacE_{T25N,S192D}. Only FLAG-K-Ras 4B_{G12V} interacted with RacE (FIG. 11D). When the interaction of K-Ras 4B and RacE was analyzed in SEC, it was found that these two proteins assemble into hetero-oligomers, similar to those identified for RasC and RacE (FIG. 11E). Furthermore, whether GTP-K-Ras 4B_{G12V} can replace GTP-RasC_{Q62L} was tested in an in vitro AKT phosphorylation assay. Indeed, purified FLAG-K-Ras 4B_{G12V} supported mTORC2 mediated AKT phosphorylation together with phospho GDP-RacE (FIG. 11F). This activation was blocked when RacE mutants that are defective in phosphorylation site (RacE_{T25N,S192A}) or binding to RasC (RacE_{T25N,S192D,E180A}) were used (FIG. 11F). These data suggested that human KRas4B is a functional homolog of Dictyostelium RasC in the regulation of mTORC2-mediated AKT phosphorylation through the hetero-oligomerization with phospho GDP-RacE.

### Example 10

### Peptide Modification and Screening

K-tetracosapeptide will be modified with N-methylation, PEGylation, cyclization, addition of β-amino acids, and substitution of D-amino acids, individually or in combination. In addition, the K-tetracosapeptide will be systematically mutagenized to identify locations of invariability.

The unmodified and modified K-tetracosapeptides will be screened for inhibition of Ras protein activity, as described above. In addition, the peptides will be tested for activity against mTORC2 activation and insulin-stimulated AKT-phosphorylation as described above.

The unmodified and modified K-tetracosapeptides will be tested for an inhibitory effect on the proliferation of K-Ras mutated cell lines, including but not limited to LS 180 (large intestine K-RAS_{G12D}), NCI-H747 (large intestine K-RAS_{G13D}), SK-CO-1 (large intestine K-RAS_{G12V}), Calu-1 (lung K-RAS_{G12C}), NCI-H1944 (lung K-RAS_{G13D}), AsPC-1 (pancreas K-RAS_{G12D}), and Capan-1 (pancreas K-RAS_{G12V}).

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A peptide, or a pharmaceutically acceptable salt or solvate thereof, comprising an amino acid sequence with at least 50% similarity to GVDDAFYTLVREIRKHKEKMSKDG (SEQ ID NO: 1).

2. The peptide of claim 1, comprising an amino acid sequence with at least 70% similarity to SEQ ID NO: 1.

3. The peptide of claim 1 or claim 2, comprising amino acid sequence of SEQ ID NO: 1.

4. The peptide of any of claims 1-3, further comprising a trafficking sequence.

5. The peptide of claim 4, wherein trafficking sequence is derived from the HIV TAT protein.

6. The peptide of claim 4 or claim 5, wherein the trafficking sequence comprises an amino acid sequence of RKKRRQRRR (SEQ ID NO: 8) or GRKKRRQRRRPQ (SEQ ID NO: 4).

7. A polynucleotide comprising a nucleic acid sequence encoding the peptide of any of claims 1-6.

8. A composition comprising the peptide of any of claims 1-6 or a nucleic acid encoding thereof.

9. The composition of claim 8, further comprising a carrier.

10. The composition of claim 8 or claim 9, further comprising a buffer.

11. A method of treating a disease or disorder comprising administering to a subject in need thereof an effective amount of the peptide of any of claims 1-6, the polynucleotide of claim 7, or the composition of any of claims 8-10.

12. The method of claim 11, wherein the disease or disorder is characterized by hyperactive K-Ras or abnormal K-Ras signaling.

13. The method of claim 11 or claim 12, wherein the disease or disorder comprises cancer or a developmental disorder.

14. The method of any of claims 11-13, wherein the disease or disorder comprises lung cancer, colorectal cancer, pancreatic cancer, ovarian cancer, breast cancer, or any combination thereof.

15. A method for modulating the activity of a Ras protein, said method comprising contacting the Ras protein with an effective amount of the peptide of any of claims 1-6, or the composition of any of claims 8-10.

16. The method of claim 15, wherein modulating the activity causes a decrease in the activity of the Ras protein.

17. The method of claim 15 or claim 16, wherein the activity comprises binding to RhoA, mTORC2 activation, insulin-stimulated AKT phosphorylation, or a combination thereof.

18. The method of any of claims 15-17, wherein the Ras protein is mutant Ras protein.

19. The method of any of claims 15-18, wherein the Ras protein is K-Ras.

20. The method of any of claims 15-19, wherein the Ras protein is K-Ras 4B.

21. The method of any of claims 15-20, wherein the Ras protein is within a biological cell.

22. The method of any of claims 15-21, wherein the biological cell is ex vivo or in an organism.


SEQUENCE LISTING (8 sequences)

SEQ ID NO: 1 - protein, 24 aa, Artificial sequence
  Synthetic
GVDDAFYTLVREIRKHKEKMSKDG

SEQ ID NO: 2 - protein, 24 aa, Artificial sequence
  Synthetic
HRATGFLEKRKDVKVGEDSYMDIK

SEQ ID NO: 3 - protein, 36 aa, Artificial sequence
  Synthetic
GRKKRRQRRRPQGVDDAFYTLVREIRKHKEKMSKDG

SEQ ID NO: 4 - protein, 12 aa, Artificial sequence
  Synthetic
GRKKRRQRRRPQ

SEQ ID NO: 5 - DNA, 20 bp, Artificial sequence
  Synthetic
actgtgggcacatacacctc

SEQ ID NO: 6 - DNA, 20 bp, Artificial sequence
  Synthetic
cggaagaaactggtgattgt

SEQ ID NO: 7 - DNA, 20 bp, Artificial sequence
  Synthetic
caatgagggaccagtacatg

SEQ ID NO: 8 - protein, 9 aa, Artificial sequence
  Synthetic
RKKRRQRRR